(19) United States
(12) Patent Application Publication
(10) Pub. No.: US 2024/0312562 A1
(43) Pub. Date: Sep. 19, 2024

(54) METHODS AND KITS FOR ENRICHING FOR POLYNUCLEOTIDES

(71) Applicant: Eclipse Bioinnovations, Inc., San Diego, CA (US)

(72) Inventors: Alexander A. Shishkin, San Diego, CA (US); Kylie An-yi Shen, San Diego, CA (US); Siarhei Manakou, San Diego, CA (US)

(21) Appl. No.: 18/250,423

(22) PCT Filed: Oct. 25, 2021
(86) PCT No.: PCT/US2021/056471

§ 371 Date: Apr. 25, 2023

Related U.S. Application Data
(60) Provisional application No. 63/105,741, filed on Oct. 26, 2020.

(51) Int. Cl. G16B 25/20; C12N 15/10; C12Q 1/6806; C12Q 1/686; C12Q 1/6869; C12Q 1/6876

(52) U.S. Cl. CPC G16B 25/20; C12N 15/1096; C12Q 1/6806; C12Q 1/686; C12Q 1/6869; C12Q 1/6876

(57) ABSTRACT

Growing demand in RNA-targeted therapies and promise of miRNA-based drugs creates a need for tools that can accurately identify and quantify miRNA:target interactions at scale. Chimeric miRNA:mRNA reads provide a direct read out of miRNA targets by capturing interaction of miRNA and targeted transcripts. In aspects described herein are methods for enriching microRNA (miRNA) targeted RNA molecules. In yet further aspects described herein are methods for enriching chimeric microRNA (miRNA)-targeted RNA molecules.

### CROSS-REFERENCE TO RELATED APPLICATIONS

This application is a National Phase of International Application No. PCT/US2021/056471, filed on Oct. 25, 2021, and published on May 5, 2022, as WO 2022/093701, which claims the benefit of U.S. Provisional Application No. 63/105,741, filed on Oct. 26, 2020, the content of which is incorporated herein by reference in its entirety.

### FIELD OF THE INVENTION

This invention relates to methods and system for enriching RNA molecules from a sample. More particularly, this invention relates to methods and systems for using Argonaute proteins to enrich a sample for chimeric microRNA molecules.

### BACKGROUND

MicroRNAs (miRNAs) represent an important class of small non-coding RNAs (sRNAs) that regulate gene expression by targeting messenger RNAs (mRNAs). miRNAs directly bind to many mRNAs to regulate their translation or stability. Thousands of miRNAs have been identified in animals and plants by cloning and deep sequencing; however, determining the targets of these miRNAs is an ongoing challenge.

### REFERENCE TO SEQUENCE LISTING

The present application is filed with a Sequence Listing in Electronic format. The Sequence Listing is provided as a file entitled EBIO.003NP_SEQLISTING_ST.25.txt, created May 23, 2024, which is approximately 4 kb in size. The information in the electronic format of the sequence listing is incorporated herein by reference in its entirety.

### SUMMARY

Some embodiments of the present disclosure relate to a method of enriching microRNA (miRNA) targeted RNA molecules. The method comprises: 1) contacting an RNA sample with target specific miRNA molecules in the presence of Argonaute 2 (Ago2) proteins to form a complex, 2) isolating the complex, 3) ligating the miRNA molecules to the RNA molecules within each complex to form chimeric RNA molecules, 4) enriching non-chimeric RNA molecules of interest and chimeric RNA molecules, or cDNA molecules thereof, with probes, 5) amplifying enriched non-chimeric RNA molecules and chimeric RNA molecules, or cDNA molecules thereof, by PCR, 6) sequencing the PCR products, and 7) identifying computationally non-chimeric RNA molecules of interest and/or chimeric RNA molecules.

Some embodiments of the present disclosure relate to a method of enriching microRNA (miRNA) targeted RNA molecules. The method comprises: 1) providing Ago2 proteins and fixing or crosslinking miRNAs and RNAs inside the Ago2 proteins to form Ago2-RNA complexes, 2) isolating Ago2-RNA complexes, 3) ligating the miRNA molecules to the RNA molecules within each Ago2-RNA complex to form chimeric RNA molecules, 4) enriching non-chimeric RNA molecules and chimeric RNA molecules of interest with probes, 5) amplifying enriched non-chimeric RNA molecules and chimeric RNA molecules by PCR, 6) sequencing the PCR products, and 7) identifying computationally chimeric RNA molecules of interest.

In some embodiments, the RNA sample is from cells or tissue. In some embodiments, the method further comprises lysing cells prior to isolating the complexes. In some embodiments, wherein contacting the RNA sample further comprises crosslinking the complex together by UV light or a chemical crosslink agent. In some embodiments, the chemical crosslink agent is selected from formaldehyde, formalin, acetaldehyde, prionaldehyde, water-soluble carbomiidides, phenylglyoxal, and UDP-dialdehyde. In some embodiments, the RNA sample comprises mRNA molecules or mRNA fragments. In some embodiments, isolating the complex is by immunoprecipitation of the complex. In some embodiments, the immunoprecipitation comprises contacting the complex with an Ago2 antibody. In some embodiments, the contacting step is followed converting associated RNA into libraries that can be subjected to high-throughput sequencing to quantify association. In some embodiments, the non-chimeric RNA molecules of interest are miRNA molecules. In some embodiments, the probes are anti-sense nucleic acid probes in a length between 10 bp and 100 bp. In some embodiments, the probes are 100% complementary to the miRNA molecules. In some embodiments, the non-chimeric RNA molecules of interest map to specific genes or 3′-UTR of genes. In some embodiments, the probes are anti-sense nucleic acid probes in a length between 10 bp and 5000 bp. In some embodiments, the cDNA molecules are formed by reverse transcribing RNA molecules into the cDNA molecules before the enriching step. In some embodiments, the probes are RNA, single stranded DNA (ssDNA), or synthetic nucleic acids, such as LNA. In some embodiments, the method further comprises digesting the Ago2 proteins prior to the enriching step. In some embodiments, the enrichment step produces about 5% to about 30% chimeric reads out of all uniquely mapped reads. In some embodiments, the enrichment step increases the proportion of chimeric reads in the library. In some embodiments, the overall chimeric read population is increased by at least 20-fold. In some embodiments, the method does not include a gel clean up step. In some embodiments, wherein omitting a gel clean up step creates a simplified high throughput of enriched miRNA. In some embodiments, the enrichment step further comprises an expression of miRNA. In some embodiments, the Ago2 is an anti-human Ago2 antibody. In some embodiments, wherein the Ago2 includes a gene selected from APP, ATG9A, BTG2, and ULK1. In some embodiments, the method further comprises immunoprecipitating RNA end repair. In some embodiments, the RNA end repair utilizes at least one of FastAP, a phosphatase that removes 5′-phosphate from RNA-DNA chimeric molecules, and T4 PNK. In some embodiments, the complexes are incubated with proteases to digest the Ago2 protein and release the ligated RNA fragments from the formed complexes. In some embodiments, the probes are selected from RNA, ssDNA, and synthetic nucleic acid. In some embodiments, the synthetic nucleic acid is LNA. In some embodiments, after the enriching step a sequencing adapter with a UMI or Randomer is ligated to the enriched and non-enriched molecules.

Some embodiments relate to a method of enriching chimeric microRNA (miRNA)-targeted RNA molecules. In some embodiments, the method comprises providing Ago2 proteins, fixing or crosslinking miRNAs and RNAs inside the Ago2 proteins to form Ago2-RNA complexes, isolating the Ago2-RNA complexes, ligating the miRNA molecules to the RNA molecules within each Ago2-RNA complex to form chimeric RNA molecules, enriching non-chimeric RNA molecules and chimeric RNA molecules of interest with probes, amplifying enriched non-chimeric RNA molecules and chimeric RNA molecules by PCR, sequencing the PCR products; and identifying computationally chimeric RNA molecules of interest. In some embodiments, the RNA molecules of interest is APP, ATG9A, BTG2, and

ULK1. In some embodiments, the fixing or crossing linking is by UV light or a chemical cross link agent. In some embodiments, the chemical crosslink agent is selected from formaldehyde, formalin, acetaldehyde, prionaldehyde, water-soluble carbomiidides, phenylglyoxal, and UDP-dialdehyde. In some embodiments, isolating the complex is by immunoprecipitation of the complex. In some embodiments, the immunoprecipitation comprises contacting the complex with an Ago2 antibody. In some embodiments, the method further comprises digesting the Ago2 proteins prior to the enriching step. In some embodiments, the enrichment step produces about 5% to about 30% chimeric reads out of all uniquely mapped reads. In some embodiments, the enrichment step increases the proportion of chimeric reads in the library. In some embodiments, the method does not include a gel clean up step. In some embodiments, wherein omitting a gel clean up step creates a simplified high throughput of enriched miRNA. In some embodiments, the enrichment step further comprises expressing miRNA. In some embodiments, the method further comprises immunoprecipitating RNA end repair. In some embodiments, the RNA end repair utilizes at least one of FastAP, a phosphatase that removes 5′-phosphate from RNA-DNA chimeric molecules, and T4 PNK.

Some embodiments relate to a method for short probe capture-based miRNA enrichment. In some embodiments, the method comprises pre-coupling ssDNA biotinylated probes to streptavidin beads to form a complex, mixing a sample of miR+adapter, mRNA+adapter, chimera miR+mRNA+adapter, the complex and a hybridization buffer, incubating the sample, the complex, and the hybridization buffer at 60° C. for 1 to 2 hours, rinsing the sample and the complex to remove background binding and to keep miR-specific molecules, eluting the complex with DNase, and sequencing the sample. In some embodiments, the ssDNA biotinylated probes are anti-sense to miRs. In some embodiments, the ssDNA biotinylated probes are 100% anti-sense to miRs. In some embodiments, the complex obtains both chimeric reads an miRNA reads.

Some embodiments relate to a method for identifying specific mRNA-miRNA binding from cells or tissues which contain RNA molecules, miRNA molecules, and Ago2 protein. In some embodiments, the method comprises crosslinking cells or tissues to link miRNA to Ago2, miRNA-mRNA to Ago2, and mRNA to Ago2, lysing cells or tissues with RNase 1 to partially fragment RNA, coupling the fragmented RNA with beads which are pre-coupled to an Ago2 antibody, washing the beads, running intermolecular ligation to form chimeric miRNA-mRNA molecules, washing the miRNA-mRNA molecules, repairing RNA ends using FastAP, DNase or T4 pNK, ligating the miRNA-mRNA molecules with a sequence adapter with UMI/randomer, digesting Ago2 protein to release RNA fragments, reverse transcribing RNA molecules to convert into cDNA, amplying the cDNA with PCR, sequencing the libraries made from the PCR, and analyzing the libraries.

These and other features, aspects, and advantages of the present disclosure will become better understood with reference to the following description and appended claims.

### BRIEF DESCRIPTION OF THE DRAWINGS

FIG. 1 is a bar graph depicting the frequency of chimeric fragments in libraries taken from different tissue types. FIG. 1 shows that chimeric rate is greater with added chimeric ligation than without (AGO2 eCLIP v. miR-eCLIP and CLEAR CLIP) and that chimeric rate with Probe Enrichment is greater than with No Enrichment. Chimeric rate is expressed as a ratio of PCR deduplicated uniquely mapped chimeric reads and a sum of counts of deduplicated uniquely mapped chimeric and non-chimeric reads. Error bars show standard deviation.

FIG. 2 is a bar graph depicting the chimeric rate of a standard eCLIP method (n=2) versus two versions of Total Chimeric miR-eCLIP method: no-gel (n=2) and with-gel (n=2).

FIG. 3 is a scatterplot depecting AGO2 correlation of IP/input enrichment in clusters of non-chimeric reads between replicate 1 of no-gel (y-axis) and replicate 1 of with-gel (x-axis) Total Chimeric miR-eCLIP assays.

FIG. 4 is a scatterplot depicting chimeric mik reads only. Shows correlation of chimeric read RPMs per each miRNA between no-gel (y-axis) and with-gel (x-axis) Total Chimeric miR-eCLIP assays.

FIG. 5 is a bar graph depiciting PCR duplication rates. PCR duplication rate based on non-chimeric mapping of reads from AGO2 eCLIP (“eCLIP”) and chimeric miR-eCLIP experiments. A comparison is made to external third party published data sets (iCLIP and CLEAR-CLIP methods).

FIG. 6 is a cartoon illustration showing generating chimeras using a modified eCLIP protocol.

FIG. 7 is a bar graph depiciting RNA peaks as a percentage from gel and no gel results. Distribution of peaks called with non-chimeric reads in Total Chimeric with-gel and no-gel experiments (n=2, replicates shown separately).

FIG. 8 is a set of four scatterplots, showing correlation of RPMs of per-miRNA non-chimeric reads (x-axis) and chimeric reads (y-axis) in two Total Chimeric.

FIG. 9 is a bar graph depicting chimeric reads in total chimeric AGO2 eCLIP. RPM of chimeric reads per top 75 miRNAs in Total Chimeric with-gel experiments.

FIG. 10 is shows fraction of chimeric reads with target portions containing a seed match for cognate miRNA (error bars show standard deviation, n=2).

FIG. 11 is a bar graph depicting fractions of chimeric reads. Shows distribution of chimeric reads from with-gel Total Chimeric experiments between intergenic and genic partitions.

FIG. 12 is a cartoon illustration of a simplified protocol of an embodiment of the disclosure.

FIG. 13 is a graph depicting read density distributions of non-chimeric and chimeric eCLIP reads with gel and no gel libraries.

FIG. 14 is a scatterplot depicting the correction between no-gel non-chimeric RPM with gel nonchimeric RPM.

FIG. 15 is a bar graph depicting chimeric reads from five miRs.

FIG. 16 is a bar graph depicting chimeric reads from miR-17 family.

FIG. 17 is a bar graph depicting chimer reads from let-7 family.

FIG. 18 is a scatterplot depicting the distribution of chimeric reads among individual miRNa specific to 5 mirs.

FIG. 19 is a scatterplot depicting the distribution of chimeric reads among individual miRNa specific to miR 17.

FIG. 20 is a scatterplot depicting the distribution of chimeric reads among individual miRNa specific to let7.

FIG. 21 is a scatterplot depicting the proportion of reads with seed matches to cognate miRNAs varies between different miRNas.

FIG. 22 is a scatterplot depicting the proportion of reads with seed matches to cognate miRNAs varies between different miRNas.

FIG. 23 is a scatterplot depicting the proportion of reads with seed matches to cognate miRNAs varies between different miRNas.

FIG. 24 is bar graph depicting the increased representation of chimeras for miRNAs of the interest among chimeric reads.

FIG. 25 is a scatterplot depicting analysis of per-miRNA read counts.

FIG. 26 is a scatterplot depicting analysis of per-miRNA read counts.

FIG. 27 is a scatterplot depicting seed matching sites for miRNA targeting.

FIG. 28 is a scatterplot depicting seed matching sites for miRNA targeting.

FIG. 29 is a scatterplot depicting enrichment of chimeric reads for ULK1.

FIG. 30 is a scatterplot depicting enrichment of chimeric reads for APP.

FIG. 31 is a scatterplot depicting enrichment of chimeric reads for ULK1.

FIG. 32 is a scatterplot depicting enrichment of chimeric reads for APP.

FIG. 33 is a graph depicting distinct peaks in chimeric read density identifying four and five actively engaged miRNA target sites in 3′ UTRs of ULK1.

FIG. 34 is a graph depicting distinct peaks in chimeric read density identifying four and five actively engaged miRNA target sites in 3′ UTRs of APP.

FIG. 35 is a scatterplot depicting DESeq2 to quantify differential gene expression upon miRNA overexpression.

FIG. 36 is a bar graph depicting 3′UTRs of downregulated miR-1 and miR-124 seed matches.

FIG. 37 is a line graph depicting miR-124 seed matching site enrichment in miR-124 over-expression experiment.

FIG. 38 is a line graph depicting miR-1 seed matching site enrichment in miR-1 over-expression experiment.

FIG. 39 is a bar graph depicting miR eCLIP targets for miR-124 transfection.

FIG. 40 is a bar graph depicting miR eCLIP targets for miR-1 transfection.

FIG. 41 is a box and whisker graph depicting miR-124 transfection.

FIG. 42 is a box and whisker graph depicting miR-1 transfection.

FIG. 43 is a schematic diagram depicting one embodiment of a protocol for enriching of chimeric RNA sequences. In this protocol, the Ago2 complexes containing miRNA and mRNA fragments are isolated, miRNA and mRNA fragments are ligated to one another to form a chimeric RNA molecule, and the chimeric molecules are then sequenced.

FIG. 44 is a flow diagram depicting the steps in the total chimeric-eCLIP protocol in one embodiment.

FIG. 45 is a schematic diagram depicting the mixture of miRNA, mRNA, and miRNA-mRNA chimeric molecules that are isolated by digesting Ago2 complexes. Total chimeric eCLIP libraries are comprised of miRNA, mRNA and miRNA-mRNA chimeric molecules. The miRNA-mRNA chimeric molecules of interest comprise approximately 1% of the final library.

FIG. 46 shows the limitations of PCR-based miRNA specific chimeric eCLIP and complexity of miRNA family members.

FIG. 47 is a flow diagram and description of an enrichment protocol for performing the probe capture-based miRNA-enrichment.

FIG. 48 is an experimental outline of a capture-based miRNA-specific experiment using anti-miRNAs probes.

FIG. 49 shows that enriched motifs in mRNA targets using probe capture-based targeted chimeric eCLIP match the reverse complement of the targeted miRNA seed sequence. The presence of the reverse complement of the miRNA seed sequence in chimeric molecules is an indication that mRNA targets are correctly identified, as miRNAs require seed sequence complementarity to bind to mRNA targets.

FIG. 50 is a flow diagram showing one embodiment of a method for performing gene-specific capture-based targeted chimeric eCLIP using RNA probes.

FIG. 51 is a diagram showing capture-based gene-specific preparation using antisense nucleic acid RNA probes.

### DETAILED DESCRIPTION

In the Summary Section above and the Detailed Description Section, and the claims below, reference is made to particular features of the disclosure. It is to be understood that the disclosure in this specification includes all possible combinations of such particular features. For example, where a particular feature is disclosed in the context of a particular aspect or embodiment of the disclosure, or a particular claim, that feature can also be used, to the extent possible, in combination with and/or in the context of other particular aspects and embodiments of the disclosure, and in the disclosure generally.

Some embodiments relate methods and system for enriching a sample for particular microRNA (miRNA) targeted RNA molecules. In some embodiments, the method includes contacting an RNA sample from a tissue or other biological source with target specific miRNA molecules in the presence of Argonaute 2 (Ago2) proteins. This will form a complex between the miRNA, target RNA, and Ago2 protein. Next the complex can be isolated away from other portions of the biological sample. The miRNA molecules and the RNA molecules in the complex can then be ligated to each other within each complex to form chimeric RNA molecules. The complexed and ligated miRNA:RNA complexes can then be enriching for non-chimeric RNA molecules of interest and chimeric RNA molecules, or cDNA molecules thereof, with probes. The enriched non-chimeric RNA molecules and chimeric RNA molecules, or cDNA molecules thereof, can then be amplified by PCR. The resulting amplicons can then be sequenced to computationally identify the chimeric and/or non-chimeric RNA molecules of interest and chimeric RNA molecules in the sample.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there is a plurality of definitions for a term herein, those in this section prevail unless stated otherwise.

“Ago2” is a member of the Argonaute (Ago) protein family. The family members are needed for miRNA-induced silencing. They bind the mature miRNA and orient it for interaction with a target mRNA. Ago family members are needed for miRNA-induced silencing. They bind to the mature miRNA and orient it for interaction with a target RNA. The miRNA binds to its targeted RNA molecules through complementary binding inside the Ago2 complex. The miRNA, its targeted RNA, and the Ago2 protein form a complex which can then be fixed or crosslinked and purified out of solution.

“LNA,” locked nucleic acid, often referred to as inaccessible RNA, is a modified RNA nucleotide in which the ribose moiety is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired and hybridize with DNA or RNA according to Watson-Crick base-pairing rules. The locked ribose conformation enhances base stacking and backbone pre-organization. This significantly increases the hybridization properties (melting temperature) of oligonucleotides.

As used herein, the term “eCLIP” broadly describes an enhanced version of the crosslinking and immunoprecipitation (CLIP) assay, and is used to identify the binding sites of RNA binding proteins (RBPs).

As used herein, the term “miR-eCLIP” broadly describes a method for identification of miRNA target sites for all expressed miRNAs and target RNA transcripts transcriptome-wide or after enrichment for miRNAs of interest or after enrichment for target transcripts of interest. Broadly speaking, the miR-eCLIP method enables precise mapping of direct miRNA-mRNA interactions transcriptome wide.

As used herein, the term “total chimeric miR-eCLIP” describes a total chimeric with gel miR-eCLIp and/or a total chimeric no gel miR-eCLIP.

As used herein, the term “miR-eCLIP+miR” describes a Total Chimeric No Gel miR-cCLIP with an added probe capture enrichment for miRNAs of interest.

As used herein, the term “miR-eCLIP+Gene” describes a Total Chimeric No Gel miR-eCLIP with an added probe capture enrichment for transcripts of a gene of interest.

As used herein, the term “miR-eCLIP+siRNA” describes a Total Chimeric No Gel miR-eCLIP with added probe capture enrichment for siRNA.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviations, per the practice in the art. Alternatively, “about” can mean a range of up to 20%, up to 10%, up to 5%, and up to 1% of a given value. Alternatively, particularly with respect to biological systems or processes, the term can mean within an order of magnitude, within 5-fold, and within 2-fold, of a value. Where particular values are described in the application and claims, unless otherwise stated the term “about” meaning within an acceptable error range for the particular value should be assumed.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a process, the term “comprising” means that the process includes at least the recited steps but may include additional steps. When used in the context of a compound, composition or device, the term “comprising” means that the compound, composition or device includes at least the recited features or components but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

All references cited herein, including but not limited to published and unpublished applications, patents, and literature references, are incorporated herein by reference in their entirety and are hereby made a part of this specification.

Where a range of values is provided, it is understood that the upper and lower limit, and each intervening value between the upper and lower limit of the range is encompassed within the embodiments.

### Methods and Uses

MicroRNAs (miRNAs) are small non-coding RNAs that regulate target genes via complementarity to messenger RNAs (mRNA), resulting in post-transcriptional repression of hundreds of mRNAs. The repertoire of miRNA targets is therefore a key determinant of the biological role of a given miRNA. Regulation via miRNA-mediated repression of gene expression has been shown to be involved in nearly every physiological system. Misregulation of miRNA biology has been implicated in a broad spectrum of diseases ranging from cancer to cardiac failure. Many miRNAs also display tissue-, cell type-, or condition-specific expression patterns and play key roles in the regulation of developmental programs. Consequently, miRNAs have become attractive tools and targets for biomedical advancements. Currently several small molecules and antisense oligos that target miRNA biogenesis as well as miRNA mimics themselves are in clinical trials as candidate therapies for diseases such as non-small cell lung cancer, keloid, chronic hepatitis C, cutaneous T-cell lymphoma and Alport's syndrome. Active research and development in the area of RNA-targeted therapies creates a need for tools that can accurately profile miRNA:mRNA target interactions in different cell cultures and tissues at scale.

Generally, miRNAs exert their repressive regulatory function by guiding the RNA-induced silencing complex (RISC) to complementary target sites in the 3′ untranslated region (UTR) of target mRNAs resulting in mRNA degradation, translation inhibition, or sequestration. Building upon this principle of sequence complementarity, dozens of algorithms have been developed to predict miRNA:mRNA interactions throughout the transcriptome. Computational approaches typically focus on a small set of key features, including sequence complementarity particularly in nucleotides 2-8 (commonly referred to as the ‘seed’ region of the miRNA), and sequence conservation across species. However, many verified targets do not meet these standard criteria, and the reliance on conservation limits detection of species-specific interactions. Experimental identification of miRNA interactions has been more challenging, and as describe below may rely on immunoprecipitation (IP) of argonaut (Ago) RISC components, followed by converting associated RNA into libraries that can be subjected to high-throughput sequencing in order to quantify association, with methods such as RNA Immunoprecipitation (RIP), Crosslinking and Immunoprecipitation (CLIP), Cross-linking and sequencing of hybrids (CLASH), CLEAR-CLIP. These assays generate chimeric miRNA:mRNA reads that originate from a ligation of a molecule of miRNA and the target RNA molecule that the miRNA is bound to. Chimeric reads link miRNA and RNA of their targets, and by this provide a snap shot of in vivo miRNA:mRNA interactions. Despite their value, practical application of chimeric reads may be limited because of a high complexity of chimeric library preparation and a low rate of chimeric reads in final libraries. CLASH and CLEAR-CLIP incorporated a dedicated step aimed at facilitating miRNA:mRNA ligation, however frequency of chimeric fragments in resulting libraries remained low (around 5%, FIG. 1).

Thousands of miRNAs have been identified in animals and plants by cloning and deep sequencing. To date, a large number of target prediction computer programs have been developed, such as TargetScan, PicTar, miRanda, PITA, and RNA22 for animal miRNA targets, and miRU and TargetFinder for plant miRNA targets. In addition, several resources have been established to systematically collect and describe both experimentally validated miRNA targets (TarBase, miRecords) and predicted miRNA targets (miRGator, MiRNAMap). However, miRNA regulation of an animal mRNA requires base pairing with only few nucleotides of the 3′-UTR region of the target mRNA; thus, a miRNA could regulate a broad range of targets, and different target prediction programs produce different results and have high false positive rates. In addition, many miRNAs are present in closely related miRNA families, complicating interpretation of loss of function studies in mammals. One caveat common to all of these studies is their inability to definitively distinguish direct from indirect miRNA-target interactions.

Some embodiments relate to a method of enriching microRNA (miRNA) targeted RNA molecules. In some embodiments, the method comprises 1) contacting an RNA sample with target specific miRNA molecules in the presence of Argonaute 2 (Ago2) proteins to form a complex, 2) isolating the complex, 3) ligating the miRNA molecules to the RNA molecules within each complex to form chimeric RNA molecules, 4) enriching non-chimeric RNA molecules of interest and chimeric RNA molecules, or cDNA molecules thereof, with probes, 5) amplifying enriched non-chimeric RNA molecules and chimeric RNA molecules, or cDNA molecules thereof, by PCR, 6) sequencing the PCR products, and 7) identifying computationally chimeric and/or non-chimeric RNA molecules of interest and chimeric RNA molecules.

Some embodiments of the present disclosure relates to a method of enriching microRNA (miRNA) targeted RNA molecules. In some embodiments, the method comprises: 1) providing Ago2 proteins and fixing or crosslinking miRNAs and RNAs inside the Ago2 proteins to form Ago2-RNA complexes, 2) isolating Ago2-RNA complexes, 3) ligating the miRNA molecules to the RNA molecules within each Ago2-RNA complex to form chimeric RNA molecules, 4) enriching non-chimeric RNA molecules and chimeric RNA molecules of interest with probes, 5) amplifying enriched non-chimeric RNA molecules and chimeric RNA molecules by PCR, 6) sequencing the PCR products, and 7) identifying computationally chimeric RNA molecules of interest.

In some embodiments, a method provided herein may be integrated during the chimeric ligation step into a method described herein to boost chimeric read production. In some embodiments, the read production may be increased by at least 2-fold. In some embodiments, the read production may be increased by at least 3-fold. In some embodiments, the read production may be increased by at least 4-fold. In some embodiments, the read production may be increased by at least 5-fold. In some embodiments, the read production may be increased by at least 6-fold. In some embodiments, the read production may be increased by at least 7-fold. In some embodiments, the read production may be increased by at least 8-fold. In some embodiments, the read production may be increased by at least 9-fold. In some embodiments, the read production may be increased by at least 10-fold.

In some embodiments, beads can be added to an embodiment described herein. In some embodiments, the beads may be approximately 1 μm in size. In some embodiments, the beads may be a magnetic bead. In some embodiments, the beads may be a superparamagnetic particle with a bound protein. In some embodiments, the bound protein may be selective for biotin. In some embodiments, the bound protein is Streptavidin. In some embodiments, the beads are streptavidin magnetic beads. In some embodiments, the beads are a dynabeads. In some embodiments, the bead is a BcMag magnetic beads. In some embodiments, the beads are monoavidin magnetic beads. In some embodiments, a simple on-bead probe can be added to an embodiment described herein. In some embodiments, the simple on-bead probe can target and enrich libraries in chimeric reads specific to one or more miRNAs of interest.

In some embodiments, the enrichment step increases proportion of chimeric reads in the library. In some embodiments, the enrichment step may produce chimeric reads out of all uniquely mapped reads of at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, or ranges including and/or spanning the aforementioned values. In some embodiments, the enrichment step may produce 7% to 28% chimeric reads out of all uniquely mapped reads.

In some embodiments, the methods described herein can omit a gel clean up step. In some embodiments, omitting the gel clean up step may create a simplified high throughput version of the method.

In some embodiments, the overall chimeric read population may be increased by at least 5-fold, 10-fold, 15-fold, 20-fold, 25-fold, 30-fold, 35-fold, 40-fold, or ranges including and/or spanning the aforementioned values, more specific for miRNAs of interest. In some embodiments, the overall chimeric read population may be increased by at least 28-fold more specific for miRNAs of interest.

In some embodiments, a method provided herein may provide a high enrichment of chimeric reads for miRNAs of interest in cell cultures. In some embodiments, a method provided herein may provide a high enrichment of chimeric reads for miRNAs of interest in tissues. In some embodiments, a method provided herein may provide a high enrichment of chimeric reads for both miRNAs of interest in cell cultures and tissues. In some embodiments, the cell culture may be from HEK293×T cell line. In some embodiments, the tissue may be from a mouse liver. In some embodiments, the cell cultures and tissues may be from a mammalian source. In some embodiments, the mammalian source is human.

Some embodiments of the present disclosure relate to a method that can definitively identify direct miRNA-target interactions with targeted RNA, mRNA or cDNA. Some embodiments relate to a method for identifying miRNAs capable of targeting a gene of interest. In some embodiments, the method comprises 1) contacting an RNA sample with target specific miRNA molecules in the presence of Argonaute 2 (Ago2) proteins to form a complex, 2) isolating the complex, 3) ligating the miRNA molecules to the RNA molecules within each complex to form chimeric RNA molecules, 4) enriching non-chimeric RNA molecules of interest and chimeric RNA molecules, or cDNA molecules thereof, with probes, 5) amplifying enriched non-chimeric RNA molecules and chimeric RNA molecules, or cDNA molecules thereof, by PCR, 6) sequencing the PCR products, and 7) identifying computationally chimeric and/or non-chimeric RNA molecules of interest and chimeric RNA molecules.

Some embodiments relate to a method for detection of miRNAs capable of targeting a gene of interest. In some embodiments, the method comprises 1) contacting an RNA sample with target specific miRNA molecules in the presence of Argonaute 2 (Ago2) proteins to form a complex, 2) isolating the complex, 3) ligating the miRNA molecules to the RNA molecules within each complex to form chimeric RNA molecules, 4) enriching non-chimeric RNA molecules of interest and chimeric RNA molecules, or cDNA molecules thereof, with probes, 5) amplifying enriched non-chimeric RNA molecules and chimeric RNA molecules, or cDNA molecules thereof, by PCR, 6) sequencing the PCR products, and 7) identifying computationally chimeric and/or non-chimeric RNA molecules of interest and chimeric RNA molecules.

Some embodiments relate to a method for mapping individual target sites along the gene transcript with high resolution. In some embodiments, the method comprises 1) contacting an RNA sample with target specific miRNA molecules in the presence of Argonaute 2 (Ago2) proteins to form a complex, 2) isolating the complex, 3) ligating the miRNA molecules to the RNA molecules within each complex to form chimeric RNA molecules, 4) enriching non-chimeric RNA molecules of interest and chimeric RNA molecules, or cDNA molecules thereof, with probes, 5) amplifying enriched non-chimeric RNA molecules and chimeric RNA molecules, or cDNA molecules thereof, by PCR, 6) sequencing the PCR products, and 7) identifying computationally chimeric and/or non-chimeric RNA molecules of interest and chimeric RNA molecules.

In some embodiments, the target RNA sample is taken from cells or tissue. Some embodiments further include lysing cells prior to isolating the complexes formed from the RNA and Ago2 proteins. During the lysing process, cells are incubated with lysis buffer and sonicated. In some embodiments, the lysing process further includes using RNase, such as RNase I, to partially fragment RNA molecules.

In some embodiments, after the miRNA and target RNA are bound into a complex with the Ago2 protein, the RNA and protein are crosslinked together by UV light and/or a chemical crosslinking agent. Exemplary suitable chemical crosslinking agents include formaldehyde; formalin; acetaldehyde; proionaldehyde; water-soluble carbodiimides (RN═C═NR′), which include 1-ethyl-3-(3-dimethylaminopropyl)-carbodiimide (EDC), 1-ethyl-3-(3-dimethylaminopropyl)-carbodiimide hydrochloride, 1-cyclohexyl-3-(2-morpholinyl-(4-ethyl) carbodiimide metho-para-toluenesulfonate (CMC), N,N′-dicyclohexylcarbodiimide (DCC) and N, N′-diisopropylcarbodiimide (DIC), and their derivatives, as well as N-hydroxysuccinimide (NHS); phenylglyoxal; and/or UDP-dialdehyde. The UV light or chemical crosslinking agent links the miRNA and target RNA to the Ago2 protein. This can preserve the RNA integrity and also the binding relationship between the miRNA and its target RNA during the purification steps.

In some embodiments, the genes for a method provided herein may include APP, ATG9A, BTG2, and ULK1. In some embodiments, these genes were selected based on their enrichment in a method provided herein. APP is a beta-amyloid precursor, transcript variant 1, full length 3583 nt. ATG9A is an autophagy related 9A, transcript variant 1, with a full length 3770 nt. BTG2 is BTG anti-proliferation factor 2, 2729 nt full transcript length. ULK1 is Unc-51 like autophagy activating kinase 1, only 2289 nt (3′-UTR+530 bp upstream of stop codon) used for probe, full transcript length is 5322 nt.

In some embodiments, the target RNA sample comprises messenger RNA (mRNA) molecules. In some embodiments, the miRNA binds to one or more mRNAs resulting in either mRNA target cleavage or translation inhibition. In animals, miRNAs usually require complementarity to a site in the 3′-UTR of an mRNA; whereas in plants, miRNA complementarity is generally within coding regions of mRNAs.

In some embodiments, isolating the RNA/Ago2 complex is done by immunoprecipitation of the complex. In some embodiments, the immunoprecipitation includes contacting the complex with an antibody that is specific for the Ago2 protein. In some embodiments, the immunoprecipitation includes incubating the crosslinked RNA sample or lysed cells with magnetic beads which are pre-coupled to a secondary antibody that binds with the Ago2 primary antibody. The beads will bind to any complexes that contain the Ago2 protein. Using a magnet, the beads along with the Ago2 complexes can be separated from the mix.

Some embodiments further include immunoprecipitated RNA end repair. After the Ago2 complexes are isolated, miRNA and its target RNA molecules are ligated together to form miRNA-target RNA chimeric molecules. Some embodiments further include repairing RNA ends using FastAP, a phosphatase that removes 5′-phosphate from RNA-DNA chimeric molecules, and T4 PNK, which convert 2′-3′-cyclic phosphate to 3′-OH that is needed for further ligation. Some embodiments further include ligating a sequencing adapter to RNA molecules; the sequencing adapter may contain a unique molecular identifier (UMI) and/or randomer to facilitate further processes, such as PCR duplicate removal.

In some embodiments, the Ago2 complexes are incubated with proteases to digest the Ago2 protein and release the ligated RNA fragments from the formed complex.

In some embodiments, the non-chimeric RNA molecules of interest are miRNA molecules within the cell. When the sequences of miRNA molecules are known, probes can be designed to specifically bind to those miRNA molecules. Such probes can specifically bind to non-chimeric miRNA molecules, as well as miRNA-target RNA chimeric molecules for enrichment. In some embodiments, the probes are anti-sense nucleic acid probes in a length between 10 bp and 100 bp. In some embodiments, the probes are a 100% complementary to the miRNA molecules and in some cases the probes can include additional sequences to better cover imprecisely processed miRNAs.

In some embodiments, the non-chimeric RNA molecules of interest are transcribed from genes or 3′ untranslated regions (UTRs) of genes. When the sequences of certain genes or 3′-UTRs of genes are known, probes can be designed to specifically bind to those genes or 3′UTRs of genes. After genes being transcribed into mRNA molecules, the mRNA sample can be mixed with specific miRNA molecules in the presence of Ago2 proteins to form a complex. The designed probes can specifically bind to non-chimeric mRNA, as well as miRNA-target mRNA chimeric molecules for enrichment. In some embodiments, the mixture of RNA molecules is reverse transcribed into cDNA molecules before adding probes. In some embodiments, the probes are anti-sense nucleic acid probes in a length of 10 bp, 20 bp, 30 bp, 40 bp, 50 bp, 60, bp, 70 bp, 80 bp, 90 bp, 100 bp, 150 bp, 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 1000 bp, 1100 bp, 1200 bp, 1300 bp 1400 bp, 1500 bp, 1600 bp, 1700 bp, 1800 bp, 1900 bp, 2000 bp, 2100 bp, 2200 bp, 2300 bp, 2400 bp, 2500 bp, 2600 bp, 2700 bp, 2800 bp, 2900 bp, 3000 bp, 3100 bp, 3200 bp, 3300 bp, 3400 bp, 3500 bp, 3600 bp, 3700 bp, 3800 bp, 3900 bp, 4000 bp, 4100 bp, 4200 bp, 4300 bp, 4400 bp, 4500 bp, 4600 bp, 4700 bp, 4800 bp, 4900 bp, 5000 bp, or ranges including and/or spanning the aforementioned values. In some embodiments, the probes are anti-sense nucleic acid probes in a length between 10 bp and 5 kb. The probes may also be between 10 bp and 1 kb, 10 bp and 500 bp, 10 bp and 250 bp, 10 bp and 100 bp, or 10 bp and 50 bp in length.

In some embodiments, the probes are RNA, single stranded DNA (ssDNA), or synthetic nucleic acids, such as a locked nucleic acid (LNA). An LNA is often referred to as inaccessible RNA and is a modified RNA nucleotide in which the ribose moiety is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired and hybridize with DNA or RNA according to Watson-Crick base-pairing rules. The locked ribose conformation enhances base stacking and backbone pre-organization. This significantly increases the hybridization properties (melting temperature) of oligonucleotides.

In some embodiments, after enriching non-chimeric RNA molecules of interest and chimeric RNA molecules with probes, a sequencing adapter with a UMI and/or Randomer is ligated to the enriched and non-enriched molecules. The resulting products are amplified by PCR, then sequenced. Through data analysis, if the sequences of miRNA molecules are known, the miRNA's target RNA can be identified. If the sequence of a gene or 3′-UTR of a gene is known, the miRNA molecules that specifically bind to the mRNA molecules or cDNA molecules can be identified and these miRNA molecules potentially can regulate the genes' function.

In embodiments that include crosslinking, the binding relation between the miRNA and its target RNA are preserved. Thus, a method according to some embodiments can definitively identify direct miRNA-target interactions.

Some embodiments are depicted in FIGS. 43-51.

FIG. 43 is a schematic diagram depicting one embodiment of a protocol for enriching chimeric RNA sequences. In this protocol, the Ago2 complexes containing miRNA and mRNA fragments are isolated, miRNA and mRNA fragments are ligated to one another to form a chimeric RNA molecule, and the chimeric molecules are then sequenced. In this embodiment, crosslink protein:RNA complexes and immunoprecipitate Ago2 complexes with miRNA and mRNA fragments inside and a portion will be crosslinked. Next, miRNA and mRNA are ligated inside Ago2 complex into chimeric (fusion) RNA molecule. Next, the sequencing adapter (with UMI/Randomer) can be ligated to all chimeric miRNA and mRNA molecules of all genes/miRNAs from a lysate (1% or less of total molecules).

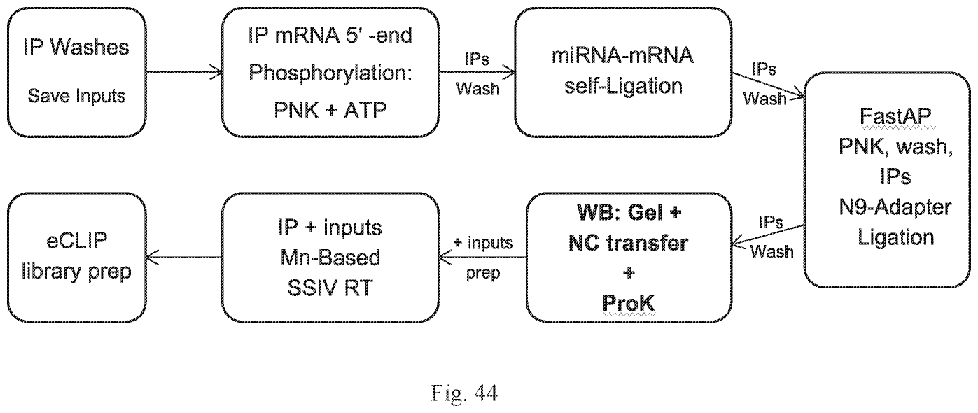

FIG. 44 is a flow diagram depicting the steps in the total chimeric-eCLIP protocol in one embodiment.

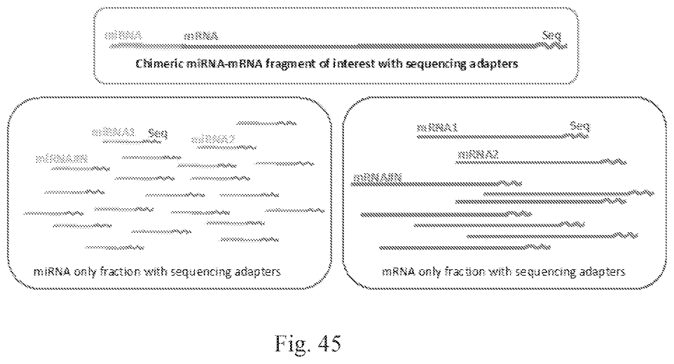

FIG. 45 is a schematic diagram depicting the mixture of miRNA, mRNA, and miRNA-mRNA chimeric molecules that are isolated by digesting Ago2 complexes. Total chimeric eCLIP libraries are comprised of miRNA, mRNA and miRNA-mRNA chimeric molecules. The miRNA-mRNA chimeric molecules of interest comprise approximately 1% of the final library. Table 1 below provides an example of the approximately 1% of the pool of molecules isolated by digesting Ago2 complexes.

TABLE 1

| | Total chimeric | Total chimeric | Total chimeric |
|---|---|---|---|
| Description | no gel Rep 1 | no gel Rep 2 | with gel |
| Initial Reads | 14,508,588 | 16,261,312 | 14,041,190 |
| # of reads containing | 986,668 | 862,194 | 1,036,946 |
| miRNA | | | |
| # of reads after removing | 258,952 | 230,058 | 457,482 |
| repetitive elements | | | |
| # of reads mapped to | 174,496 | 153,765 | 240,480 |
| the genome | | | |
| # of reads after removing | 168,156 | 146,968 | 205,212 |
| PCR duplicates | | | |
| PCR duplication rate | 3.63% | 4.42% | 14.67% |
| % chimeric reads | 1.16% | 0.9% | 1.46% |

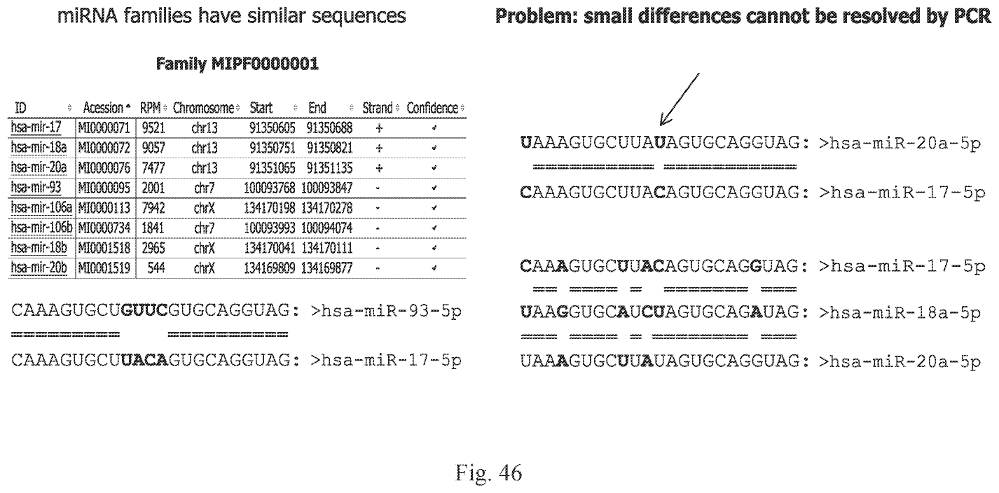

FIG. 46 shows the limitations of PCR-based miRNA specific chimeric eCLIP and complexity of miRNA family members.

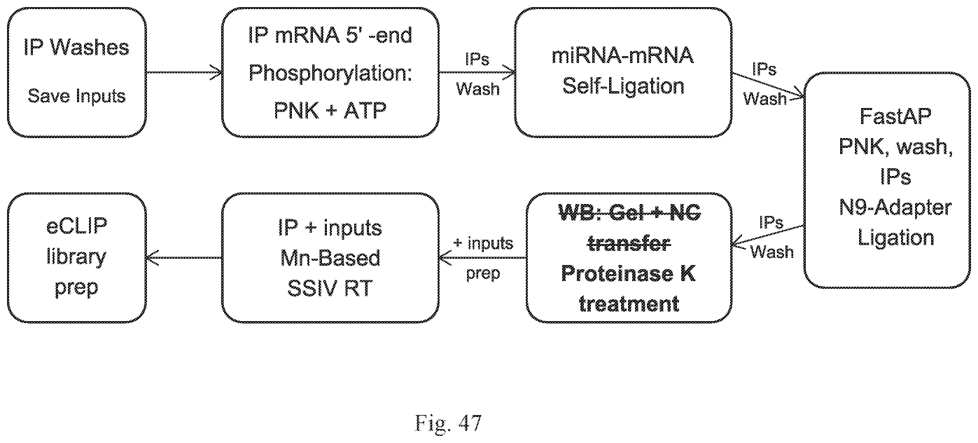

FIG. 47 is a flow diagram and description of an enrichment protocol for performing the probe capture-based miRNA-enrichment. In this embodiment, the first step includes a pre-coupling ssDNA biotinylated probes (anti-sense to miRs) to Streptavidin beads. The second step includes mixing sample (miR+adapter, mRNA+adapter, chimeric miR+mRNA+adapter)+beads with coupled probes+hybridization buffer, incubate at 60° C. for 1-2 hours (see WO2019/078909 for acceptable buffers). Rinse to remove non-specifically bound molecules. The third step includes eluting from beads (with DNase). The fourth step includes finishing library preparation, sequencing, and analyzing.

Some embodiments provide for a method for a probe capture-based miRNA enrichment chimeric eCLIP uses probes antisense to the miRNA of interest. An miRNA of interest can be enriched using anti-sense nucleic acid probes, resulting in a library containing miRNA-mRNA chimeric reads and miRNA reads. In some embodiments, the probe-based capture can be used for miRNA- or siRNA-specific chimeric-eCLIP to get all reads (including chimeric) for one or many full or partial miRNAs/siRNAs. In some embodiments, probes can be nucleic acid probes (RNA, ssDNA, LNA, etc) or any other similar molecules (including chemical analogs of RNA or ssDNA), which will allow hybridization and selection/enrichment from solution. In some embodiments, probes can be 100% anti-sense match to miRNA/siRNA or cover miRNA+/−extra sequence (for e.g., to better cover imprecisely-processed miRNAs). In some embodiments, RNA molecules for miRNA/miRNAs of interest can be captured from mixture of all molecules using anti-sense probes to obtain both chimeric reads and miRNA reads. Someone experienced in the field can easily enrich using probes anti-sense to cDNA or probes to ligated cDNA (just downstream of library prep protocol). In some embodiments, for probe capture-based miRNA enrichment can use RNA molecules as the template and ssDNA-biotinylated probes anti-sense to miRNA/siRNA of interest (oligos). In some embodiments, some siRNAs, can be ligated to mRNA/RNA targets that are not classic RNAs. For example, they are analogs of nucleic acids.

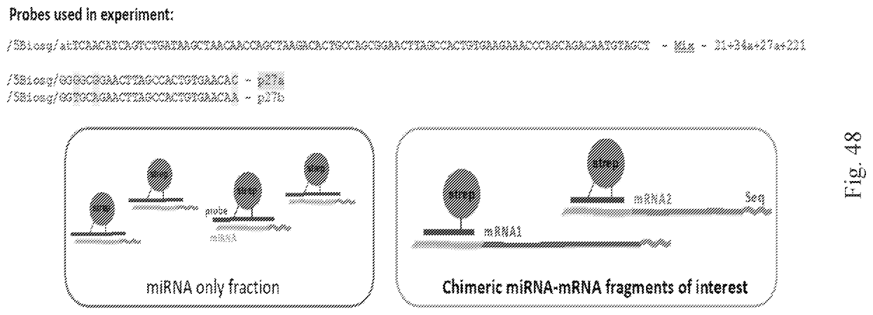

FIG. 48 illustrates an experimental outline of a capture-based miRNA-specific experiment using anti-miRNAs probes. In some embodiments, the experimental setup includes 10 million crosslinked HEK293×T cells were lysed in 1 mL of eCLIP lysis buffer, Ago2-mediated complexes were immunoprecipitated using 100 μL of anti-mouse Dynabeads and 10 μg of Ago2 antibodies (EclipseBio), total chimeric was performed as well as probe capture-based miRNA enrichment.

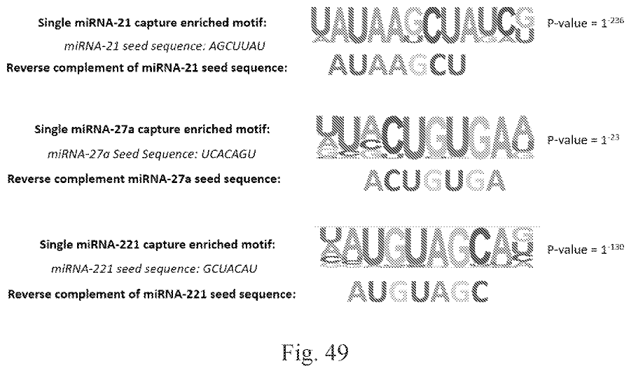

FIG. 49 shows that enriched motifs in mRNA targets using probe capture-based targeted chimeric eCLIP match the reverse complement of the targeted miRNA seed sequence. The presence of the reverse complement of the miRNA seed sequence in chimeric molecules is an indication that mRNA targets are correctly identified, as miRNAs require seed sequence complementarity to bind to mRNA targets.

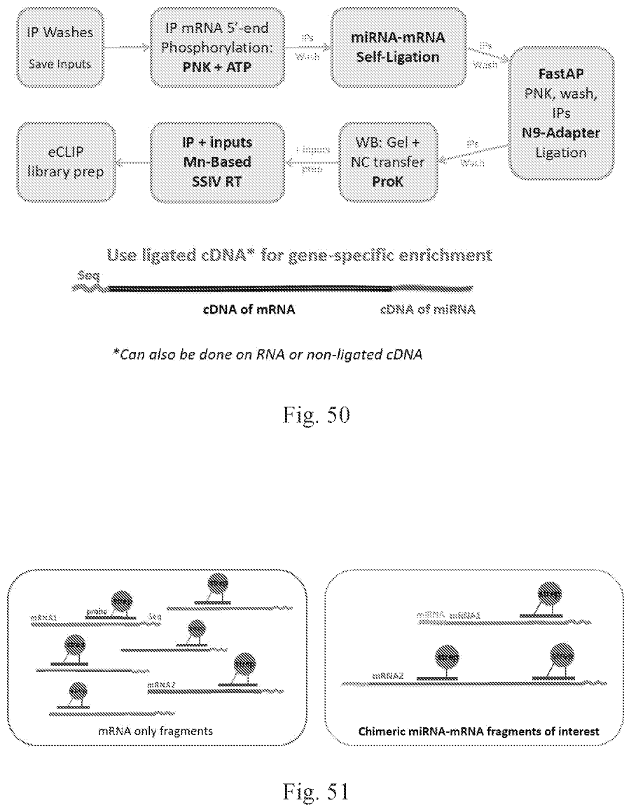

FIG. 50 illustrates a flow diagram showing one embodiment of a method for performing gene-specific capture-based targeted chimeric eCLIP using RNA probes.

FIG. 51 is a diagram showing capture-based gene-specific preparation using antisense nucleic acid RNA probes. In some embodiments, the gene-specific probe capture-based chimeric-eCLIP obtains chimeric reads as well as mRNA reads for the genes of interest. In some embodiments, the enrichment is performed by capturing RNA or cDNA or ligated cDNA molecules for gene of interest (or 3′-UTR of gene/genes of interest) from mixture using anti-sense nucleic acid probes [short or long (10-5 kb) RNA, ssDNA, synthetic nucleic acids (LNA, etc.)]. In some embodiments, the chimeric molecules comprise 1% or less of the molecules isolated by digesting Ago2 complexes.

### Kits

Also provided by this disclosure are kits for practicing the methods as described herein. A subject kit may contain one or more of particular miRNA molecules, ligase, Ago2 protein, anti-Ago2 antibodies, probed, beads, and labeled antibodies which bind to the anti-Ago2 antibodies, or a combination thereof. In some embodiments, the kit may comprise gel clean up materials. In some embodiments, the kit does not include gel clean up materials. In some embodiments, the kit may include materials to isolate RNA from cells or tissues. In some embodiments, the kit may include a chemical crosslinking agent. In some embodiments, the kit comprises a protease.

The components of the kit may be combined in one container, or each component may be in its own container. For example, the components of the kit may be combined in a single reaction tube or in one or more different reaction tubes. Further details of the components of this kit are described above. The kit may also contain other reagents described above and below that are not essential to the method but nevertheless may be employed in the method, depending on how the method is going to be implemented.

In addition to above-mentioned components, the subject kits may further include instructions for using the components of the kit to practice the subject methods, i.e., to provide instructions for sample analysis. The instructions for practicing the present method may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

Embodiments also include kits containing the components required to perform the methods and assays described herein. For example, the kit may contain particular miRNA molecules, ligase, Ago2 protein, anti-Ago2 antibodies, and labeled antibodies which bind to the anti-Ago2 antibodies.

### EXAMPLES

The following examples are given for the purpose of illustrating various embodiments of the disclosure and are not meant to limit the present disclosure in any fashion. One skilled in the art will appreciate readily that the present disclosure is well adapted to carry out the objects and obtain the ends and advantages mentioned, as well as those objects, ends and advantages inherent herein. Changes therein and other uses which are encompassed within the spirit of the disclosure as defined by the scope of the claims will occur to those skilled in the art.

### Example 1

This example describes one embodiment of a method for identifying specific mRNA-miRNA binding from cells or tissues, which contain RNA molecules, miRNA molecules, and Ago2 protein.

In the first step: Crosslink cells or tissues to link miRNA to Ago2, miRNA-mRNA to Ago2, and mRNA to Ago2-all inside the Ago2 complex.

In the second step: Lyse cells (lysis buffer and sonication), RNase treat (RNase 1) to partially fragment RNA (mRNA fragmentation), and couple to beads which are pre-coupled to an Ago2 antibody (Immunoprecipitation of Ago2 protein).

In the third step: Perform washes to remove background.

In the fourth step: Treat RNA ends to support step 5 (intermolecular ligation): 5′-PNK-Phosphotase-minus were used to only phosphorylate 5′-RNA ends (both miRNA and mRNA). This enzyme is not “opening” 3′-RNA ends.

In the fifth step: Run intermolecular ligation to form chimeric miRNA-mRNA molecules.

In the sixth step: Perform strong washes to remove background.

In the seventh step: Repair RNA ends using FastAP, DNase and T4 PNK, leaving 3′-OH that is needed for ligation. Perform any additional washes.

In the eight step: Ligate sequencing adapter with UMI/randomer.

In the ninth step, part one: Run gels to clean chimeric and non-chimeric RNA fragments crosslinked to Ago2 protein.

In the ninth step, part two: Digest Ago2 protein to release RNA fragments. Clean RNA fragments or enrich for needed RNA fragments with probes, if applicable. When the sequences of certain miRNA molecules are known, probes are designed to specifically bind to those miRNA molecules. Such probes can specifically bind to non-chimeric miRNA molecules, as well as miRNA-mRNA chimeric molecules for enrichment.

In the tenth step: Reverse transcribe RNA molecules to convert into cDNA.

In the eleventh step: When the sequences of certain genes or 3′-UTR of genes are known, probes can be designed to specifically bind to transcripts of those genes or 3′-UTR of a gene transcript. Enrich for needed cDNA with probes, if applicable.

In the twelfth step: Perform 2^{nd} adapter ligation with UMI to enriched and non-enriched molecules.

In the thirteenth step: PCR amplify and clean up libraries for sequencing.

In the fourteenth step: Sequence the libraries made of the PCR products.

In the fifteenth step: Data analysis. The data analysis can comprise the following: A. Trim N10 UMIs from the 5′ ends of R1 reads and save the UMI sequences in the read names to be utilized in subsequent steps. B. Trim N9 UMIs from the 5′ ends of R2 reads and append these UMI sequences to the N10 UMI sequence within the read names in R1 reads. C. Trim 3′ sequencing adapters and remove reads less than 18 bp in length. D. Trim 9 nucleotides from the 3′ ends of R1 reads (this removes potential UMI sequence). E. “Reverse map” mature miRNA sequences (downloaded from Mirbase) to reads. F. Filter miRNA-read alignments on 2 criteria: prioritize hits with the fewest number of mismatches and prioritize+strand alignments. G. For each read, identify sequences flanking the miRNA alignments. Remove flanking sequences that are less than 18 bp in length. H. Map reads flanking miRNA alignments to the reference genome. I. Remove PCR duplicates by utilizing UMI sequences from the read names and mapping positions. J. Annotate each chimeric read alignment with the name of the aligned miRNA, as well as the gene and transcript information from GENCODE. The following priority hierarchy is used to define the final annotation of overlapping features: protein coding transcript (CDS, UTRs, intron), followed by non-coding transcripts (exon, intron).

If the purpose of the experiment is designed to identify mRNA targets for known miRNA, such mRNA targets will be identified following the steps described herein. Similarly, if the purpose of the experiment is designed to identify what miRNA molecules target known genes or 3′-UTR of genes, such miRNA molecules will be identified following the steps described herein.

### Example 2

### Cell Culture

Human HEK293×T cells were acquired from ATCC. Cells were cultured in DMEM media (GIBCO) with 10% FBS 1% penicillin/streptomycin and grown at 37° C. in 5% CO₂. Cells were routinely tested with MycoAlert PLUS (Lonza) for myco-plasma contamination.

miR-eCLIP

eCLIP was performed in HEK293×T cells as previously described in detail (Van Nostrand et al., 2016 & 2017) but was modified to enhance chimera formation for chimeric-eCLIP, described below. 15 million cells were UV crosslinked (254 nm, 400 mJ/cm²) on ice, cells spun down, supernatant removed, and washed with cold phosphate buffered saline. Cell pellets were flash frozen on dry ice and stored at −80° C. Lysis was performed in eCLIP lysis buffer, followed by sonication and digestion with RNase I (Ambion). Immunoprecipitation of AGO2-RNA complexes was achieved with a primary mouse monoclonal Ago2 antibody (eIF2C2 (4F9) Santa Cruz, 4° C. overnight) using magnetic beads pre-coupled to the secondary antibody (M-280 Sheep Anti-Mouse IgG Dynabeads, ThermoFisher 11202D). 2% of each immunoprecipitated (IP) sample was saved as Input control. To phosphorylate the cleaved mRNA 5′-ends, beads were washed and treated with T4 polynucleotide kinase (PNK, 3′-phosphatase minus, NEB) and 1 mM ATP. Chimera ligation was performed on-bead at room temperature for one hour with T4 RNA Ligase I (NEB) and 1 mM ATP in a 150 μl total volume. After dephosphorylation with alkaline phosphatase (FastAP, Thermo Fisher) and T4 PNK (NEB), a barcoded adapter was ligated to the 3′-ends of the mRNA fragments (T4 RNA Ligase, NEB). Total chimeric-eCLIP IP samples were then decoupled from beads and along with input samples, were run on 4%-12% Bis-Tris protein gels and transferred to nitrocellulose membranes. The region corresponding to bands at the appropriate Ago2 protein size plus 75 kDa was excised and treated with Proteinase K (NEB) to isolate RNA. RNA was column purified (Zymo) and reverse transcribed with SuperScript IV Reverse Transcriptase (Invitrogen), 3 mM manganese chloride, and 0.1 M DTT; then treated with ExoSAP-IT (Affymetrix) to remove excess oligonucleotides. A 5′ Illumina DNA adapter (/5Phos/NNNNNNNNNNAGATCGGAAGAGCGTCGTGT/3SpC3-SEQ ID NO: 1) was ligated to the 3′-end of cDNA fragments with T4 RNA Ligase (NEB) and after on-bead cleanup (Dynabeads MyOne Silane, ThermoFisher), qPCR was performed on an aliquot of each sample to identify the proper number of PCR cycles. The remainder of the sample was PCR amplified with barcoded Illumina compatible primers (Q5, NEB) based on qPCR quantification and size selected using AMPure XP beads (Beckman). Libraries were quantified using Agilent4200 TapeStation and sequenced on the Illumina Nova Seq 6000 platform to a depth of approximately >8 million reads.

Probe-Based miRNA Capture

Samples were directly treated with Proteinase K in place of the SDS-PAGE and membrane transfer steps described above. Biotinylated DNA probes designed (reverse complement) to the miRNA of interest (IDT) were then hybridized (500 picomoles per sample), washed on Silane beads, and treated with DNase (Life Technologies). The remaining reverse transcription and library preparation steps were then performed as described above.

### Probe-Based Gene Capture

Samples were directly treated with Proteinase K in place of the SDS-PAGE and membrane transfer described above. Reverse transcription and cDNA adapter ligation steps were performed as above. Prior to PCR amplification, gblocks Gene Fragments (IDT) designed for the gene of interest were amplified to generate dsDNA templates. Biotinylated RNA probes were generated using T7 RNA Polymerase and biotinylated nucleotides. The biotinylated probes were coupled to streptavidin beads (10 μg per sample) and following denaturation of chimeric molecules, hybridized for one hour at 50° C. Beads were washed, genes-specific probes degraded, and enriched DNA fragments eluted from beads. The remaining PCR amplification and library preparation steps were then performed as described above.

### Example 3

Table 2 below shows that the number of usable chimeric reads is low. particularly for single-miRNA capture samples. The number of usable chimeric reads refers to the number of reads after mapping to the human genome and removing PCR duplicates.

TABLE 2

| | | | # of reads after | # of reads | # of reads | | % chimeric reads |
|---|---|---|---|---|---|---|---|
| | | # of reads | removing | mapped | after | PCR | overlapping at |
| | Initial # | containing | repetitive | to the | removing PCR | duplication | least 1 enriched |
| Description | of reads | miRNA | elements | genome | duplicates | rate | Ago2 peak |
| Total chimeric rep 1 | 17,907,015 | 1,332,382 | 300,303 | 195,849 | 190,896 | 2.5% | 22.0% |
| Total chimeric rep 2 | 16,456,155 | 995,000 | 221,122 | 143,335 | 139,925 | 2.4% | 17.8% |
| Experiment 2, enrichment rep 1 | 19,748,878 | 1,919,172 | 364,914 | 211,206 | 185,808 | 12.0% | 11.9% |
| Experiment 2, enrichment rep 2 | 21,296,519 | 1,744,354 | 339,268 | 190,952 | 162,845 | 14.7% | 10.3% |
| Supernatant of experiment 2 rep 1 | 17,805,597 | 1,427,002 | 408,920 | 243,424 | 231,324 | 5.0% | 18.4% |
| Supernatant of experiment 2 rep 2 | 23,390,450 | 1,513,804 | 457,786 | 267,870 | 247,805 | 7.5% | 15.2% |
| Single miRNA capture: miR-21 | 13.100,197 | 260,312 | 60,332 | 24,155 | 19,343 | 19.9% | 11.0% |
| Single miRNA capture: miR-27a | 13,326,181 | 183,288 | 53,582 | 21,492 | 18,466 | 14.1% | 9.7% |
| Single miRNA capture: miR-221 | 13,644,131 | 269,348 | 98,416 | 39,200 | 31,551 | 19.5% | 10.7% |
| Supernatant of miR-21 capture | 15,536,853 | 332,884 | 123,771 | 48,177 | 46,948 | 2.6% | 9.9% |
| Supernatant of miR-27a capture | 13,438,947 | 301,476 | 112,452 | 37,835 | 37,087 | 2.0% | 9.4% |
| Supernatant of miR-221 capture | 14,199,349 | 263,628 | 99,856 | 38,963 | 38,208 | 1.9% | 9.4% |

### Example 4

Table 3 below shows that targeted miRNAs are enriched in probe capture-based samples over total chimeric samples and that targeting multiple miRNAs gives a higher percentage of correct targets than targeting a single miRNA. When targeting a single miRNA, 15-56% of chimeric reads contain the correct targeted miRNA. When targeting 6 different miRNAs within the same sample, 83-85% of chimeric reads contain one of the targeted miRNAs. In all samples, the targeted miRNA reads are enriched in the probe capture-based samples over the total chimeric samples by at least 20-fold.

TABLE 3

| | | Log2 fold change |
|---|---|---|
| Description | % correct target | vs. total chimeric |
| Total chimeric rep 1 | 4.0% | NA |
| Total chimeric rep 2 | 4.1% | NA |
| Experiment 2, enrichment rep 1 | 85.7% | 4.45 |
| Experiment 2, enrichment rep 2 | 83.3% | 4.32 |
| Supernatant of experiment 2 rep 1 | 17.0% | 2.12 |
| Supernatant of experiment 2 rep 2 | 24.9% | 2.57 |
| Single miRNA capture: miR-21 | 20.8% | 6.65 |
| Supernatant of miR-21 capture | 0.5% | 1.17 |
| Single miRNA capture: miR-27a | 15.7% | 5.53 |
| Supernatant of miR-27a capture | 2.7% | 2.97 |
| Single miRNA capture: mIR-221 | 56.6% | 4.94 |
| Supernatant of miR-221 capture | 0.9% | −1.09 |

### Example 5

Table 4 below shows experimental details and summary of results for miRNA probe-based capture experiment.

TABLE 4

| Description | Summary of Results |
|---|---|
| Three different individual miRNAs (1/tube) | miRNA-21: over 100-fold (2^{6.65}) enrichment |
| were enriched in three independent | of miR-21 chimeric reads in miR-21 capture |
| experiments: miR-21, miR-27a, miR-221 | vs. total chimeric |
| | miRNA-27a: 46.2-fold (2^{5.53}) enrichment |
| | miRNA-221: 30.7-fold (2^{4.94}) enrichment |
| Repeated twice: Enrichment of 6 individual | Experiment 1: 21.9-fold (2^{4.45}) enrichment |
| miRNAs at once (6/tube): | of the 6 targeted miR chimeric reads in |
| miR-27a, miR-27b, miR-221, miR-222, | capture vs. total chimeric |
| miR-34a, and miR-21 | Experiment 2: 20-fold (2^{4.32}) enrichment |

### Example 6

Table 5 and 6 show that probe capture-based miRNA enrichment chimeric eCLIP can be used to study miRNA families.

TABLE 5

| | Usable | miR-27a-3p | miR-27b-3p |
|---|---|---|---|
| Description | Chimeric Reads | reads | reads |
| Single miRNA | 18,466 | 2,904 | 3,582 |
| capture: miR-27a | | | |

For example, miR-27a successfully catching miR-27b: >hsa-miR-27a-3p MIMAT0000084 UUCACAGUGGCUAAGUUCCGC (SEQ ID NO: 2), >hsa-miR-27b-3p MIMAT0000419 UUCACAGUGGCUAAGUUCUGC (SEQ ID NO: 3).

TABLE 6

| | Usable | miR-221-3p | miR-222-3p |
|---|---|---|---|
| Description | Chimeric Reads | reads | reads |
| Single miRNA | 31,551 | 17,843 | 1,034 |
| capture: miR-221 | | | |

For example, miR-221 successfully catching miR-222: >hsa-miR-221-3p MIMAT0000278 AGCUACAU-UGUCUGCUGGGUUUC (SEQ ID NO: 4), >hsa-miR-222-3p MIMAT0000279 AGCUACAUCUGGCUACUGGGU (SEQ ID NO: 5).

### Example 7

This example illustrates a gene-specific probe description and protocol for performing enrichment for genes of interest. Probes are typically nucleic acid probes (RNA, ssDNA, LNA, etc) or any other similar molecules (including chemical analogs of RNA or ssDNA), which will allow hybridization and selection/enrichment from solution. For gene-specific probe capture-based chimeric eCLIP we enriched using cDNA molecules (with attached adapters) as templates and RNA-biotinylated anti-sense to cDNA of gene/genes of interest as probes. Some siRNAs, ligated to mRNA/RNA targets technically are not classic RNAs-analogs of nucleic acids. Someone experienced in the field can easily enrich using probes anti-sense to RNA or probes anti-sense to cDNA (downstream of library preparation protocol)

Short Probe Capture-Based miRNA Enrichment Protocol:

First, pre-couple ssDNA biotinylated probes (anti-sense to mRNA) to Streptavidin beads (Dynabeads).

Second, mix sample (miR+adapter, mRNA+adapter, chimeric miR+mRNA+adapter)+beads with coupled probes+hybridization buffer (see WO2019078909A2 for buffers), incubate at 60° C. for 1-2h. Rinse to remove background binding and to keep mRNA/RNA-specific molecules.

Third, elute from beads (with DNase).

Fourth, finish library preparation, sequence, and analyze.

### Example 8

Table 7 below shows that the number of usable chimeric reads is low for capture-based gene-specific chimeric cCLIP.

TABLE 7

| | | | # of reads after | # of reads | # of reads after | |
|---|---|---|---|---|---|---|
| | | # of reads | removing | mapped | removing PCR | PCR |
| | Initial | containing | repetitive | to the | duplicates | duplication |
| Description | reads | miRNA | elements | genome | (usable reads) | rate |
| Non-targeted control rep 1 | 31,997,526 | 1,271,074 | 424,567 | 208,774 | 204,403 | 2.09% |
| Non-targeted control rep 2 | 30,360,378 | 1,078,434 | 377,556 | 188,296 | 184,547 | 1.99% |
| Gene-capture: | 34,962,569 | 1,605,356 | 423,039 | 204,652 | 178,753 | 12.66% |
| ULK1, APP, | | | | | | |
| BTG2, and ATG9A | | | | | | |
| Single capture: | 29,235,515 | 1,108,802 | 344,370 | 163,024 | 134,898 | 17.25% |
| ULK1 3′UTR | | | | | | |

### Example 9

Table 8 shows that targeted genes are enriched compared to non-targeted controls. Chimeric reads containing the targeted mRNA were enriched in the gene-specific capture-based samples over the non-targeted control sample by at least 4-fold.

TABLE 8

| | | ULK1 fold | | APP fold | | BTG2 fold | | ATG9A fold |
|---|---|---|---|---|---|---|---|---|
| | ULK1 | change vs. | APP | change vs. | BTG2 | change vs. | ATG9A | change vs. |
| | chimeric | average | chimeric | average | chimeric | average | chimeric | average |
| Description | RPM | control | RPM | control | RPM | control | RPM | control |
| Non-targeted | 235 | NA | 259 | NA | 680 | NA | 157 | NA |
| control rep 1 | | | | | | | | |
| Non-targeted | 168 | NA | 331 | NA | 569 | NA | 70 | NA |
| control rep 2 | | | | | | | | |
| Gene capture: | 2652 | 13.2 | 3530 | 12 | 16727 | 26.8 | 470 | 4.1 |
| ULK1, APP, | | | | | | | | |
| BTG2, and ATG9A | | | | | | | | |
| Single capture: | 5812 | 28.9 | 259 | 0.9 | 689 | 1.1 | 82 | 0.7 |
| ULK1 3′UTR | | | | | | | | |

### Example 10

Table 9 shows that a higher percentage of chimeric reads overlap with enriched Ago2 peaks in gene-specific chimeric than in the supernatant.

TABLE 9

| | | | # of reads after | # of reads | # of reads | | % chimeric reads |
|---|---|---|---|---|---|---|---|
| | | # of reads | removing | mapped | after | PCR | overlapping at |
| | Initial | containing | repetitive | to the | removing PCR | duplication | least one enriched |
| Description | reads | miRNA | elements | genome | duplicates | rate | Ago2 peak |
| Single gene capture: APP rep 1 | 14,011,452 | 1,063,674 | 109,332 | 61,945 | 51,153 | 17.4% | 37.6% |
| Single capture: ULK1 3′UTR rep 1 | 11,405,292 | 815,774 | 114,546 | 68,874 | 35,706 | 48.2% | 26.9% |
| Supernatant of APP gene capture rep 1 | 19,673,894 | 1,361,708 | 333,032 | 222,699 | 216,482 | 2.8% | 21.9% |
| Supernatant of ULK1 3′UTR capture rep 1 | 21,070,869 | 1,462,854 | 354,571 | 236,608 | 230,657 | 2.5% | 21.4% |
| Single gene capture: APP rep 2 | 15,933,728 | 832,410 | 105,494 | 65,763 | 53,428 | 18.8% | 41.7% |
| Single capture: ULK1 3′UTR rep 2 | 8,592,911 | 392,578 | 65,367 | 40,703 | 27,960 | 31.3% | 27.9% |
| Supernatant of APP gene capture rep 2 | 16,044,500 | 862,174 | 222,854 | 148,636 | 145,309 | 2.2% | 20.0% |
| Supernatant of ULK1 3′UTR capture rep 2 | 15,674,939 | 842,218 | 216,839 | 145,159 | 141,558 | 2.5% | 19.8% |
| Single gene capture: APP rep 3 | 16,222,480 | 722,264 | 171,403 | 59,984 | 19,241 | 67.9% | 19.8% |
| Single capture: ULK1 3′UTR rep 3 | 13,104,162 | 227,332 | 76,248 | 29,477 | 7,753 | 73.7% | 15.7% |
| Supernetart of APP capture rep 3 | 12,380,279 | 189,376 | 74,368 | 28,934 | 28,381 | 1.9% | 9.4% |
| Supernatant of ULK1 3′UTR capture rep 3 | 12,186,162 | 177,272 | 69,574 | 26,760 | 26,230 | 2.0% | 9.3% |

### Example 11

Table 10 shows that gene-specific capture-based samples give a low percentage of the correct target, but high enrichment vs. total chimeric. The percentage of reads containing the targeted gene is 2-7%, but the number of chimeric reads containing the targeted mRNA is highly enriched in capture-based samples over total chimeric samples.

TABLE 10

| | % correct | Log2 fold change |
|---|---|---|
| Description | target | vs. total chimeric |
| Single gene capture: APP rep 1 | 2.59% | 6 |
| Single gene capture: APP rep 2 | 2.48% | 6.4 |
| Supernatant of APP capture rep 1 | 0.03% | −0.4 |
| Supernatant of APP capture rep 2 | 0.03% | 0 |
| Single gene capture: APP rep 3 | 2.31% | 3.6 |
| Supernatant of APP capture rep 3 | 0.02% | −1. |
| Single capture: ULK1 3′UTR rep 1 | 6.91% | 8.1 |
| Single capture: ULK1 3′UTR rep 2 | 5.41% | 8 |
| Supernatant of ULK1 3′UTR rep 1 | 0.02% | −0.6 |
| Supernatant of ULK1 3′UTR rep 2 | 0.02% | −0.4 |
| Single capture: ULK1 3′UTR rep 3 | 5.06% | 8.3 (315-fold) |
| Supernatant of ULK1 3′UTR rep 3 | 0.01% | −0.5 |

### Example 12

Table 11 is a summary of results from gene-specific capture-based enrichment experiments.

TABLE 11

| Description | Summary of results |
|---|---|
| Experiment #1: 3 full-length mRNAs and | All mRNAs were enriched 4.1-26.8-fold |
| one 3′-UTR of another mRNA (APP, BTG2, | for chimeric RPM of targeted gene in capture |
| ATG9A and 3′-UTR of ULK) were enriched | samples vs. chimeric RPM of targeted gene |
| in a single experiment to find miRNAs | in control samples (average values of 2 |
| binding those mRNA/3′-UTR mRNA. | control samples) |
| Experiment #2: Enrichment of single | Enrichment for ULK1 3′-UTR mRNA was |
| individual 3′-UTR of ULKI mRNA (part of | 28.9-fold for capture vs. control samples |
| mRNA) | |
| Experiments #3, #4, #5 (3 biological | APP mRNA was enriched 48.5-84.4-fold |
| replicates): full-length APP was enriched | (2^{5.6}-2^{6.4}-fold) in gene-specific capture vs. |
| using anti-sense mRNA probes. | supernatant |
| Experiments #6, #7, #8 (3 biological | Enrichment for ULK1 3′-UTR mRNA was |
| replicates): 3′-UTR of ULK1 mRNA was | 256-315-fold (2⁸-2^{8.3}-fold) for capture vs. |
| enriched using anti-sense probes to | control sample |
| partial mRNA (3′-UTR). | |

Note:
full genes are “near full-genes”, ~90-99.9% of full length

### Example 13

Table 12 shows that approximately 100 miRNAs are found to be bound to APP and ULK1.

TABLE 12

| | # of reads | # of miRNAs |
|---|---|---|
| | after removing | bound to |
| Description | PCR duplicates | targeted gene |
| Single gene capture: APP rep 1 | 51,153 | 133 |
| Single capture: ULK1 3′UTR rep 1 | 35,706 | 109 |
| Single gene capture: APP rep 2 | 53,428 | 122 |
| Single capture: ULK1 3′UTR rep 2 | 27,960 | 92 |

### Example 14

It was found that miRNAs with shared seed sites (members of one seed family) often co-target the same target sites. Sequencing technology is well suited to address quantitative biological questions, such as characterizing gene expression with RNA-seq, so it was reasoned that count of chimeric reads may also provide a quantitative metric predictive of the impact that an miRNA has on expression of a target. It was validated this assumption using a standard miRNA mimic transfection paradigm and showed that chimeric read count provides a quantitative metric that correlates with the strength with which targets are repressed on RNA level following miRNA overexpression.

The unique insight in CLASH methods (that chimeric fragments that directly link miRNA and target within the same sequencing read unambiguously identify miRNA targets) with the methodological improvements in eCLIP to develop novel technologies that enable deep profiling of miRNA targets was of interest to determine how to combine these two methods.

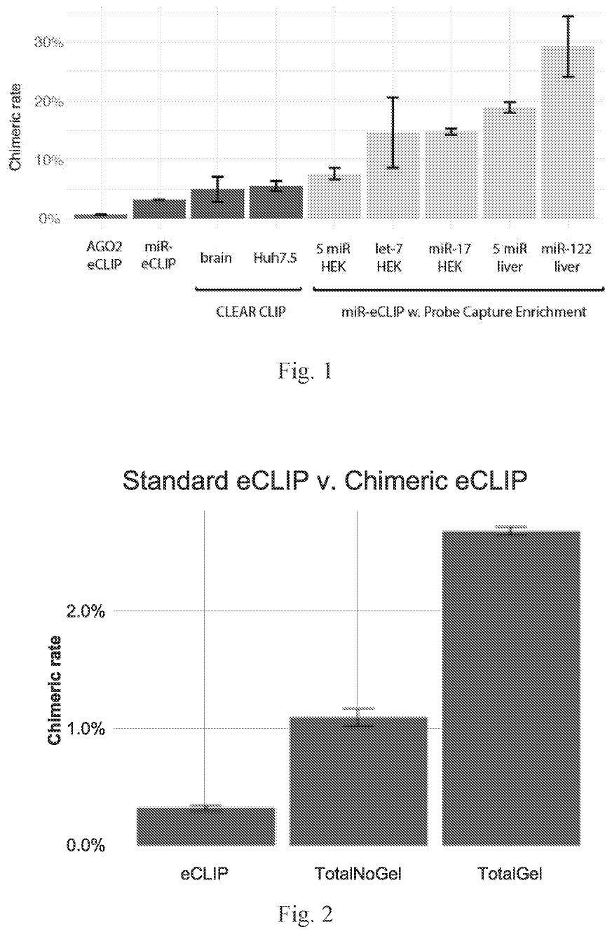

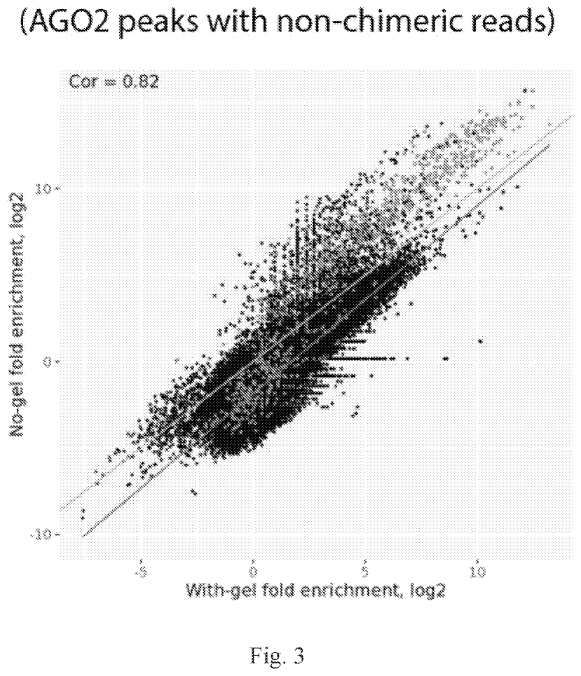

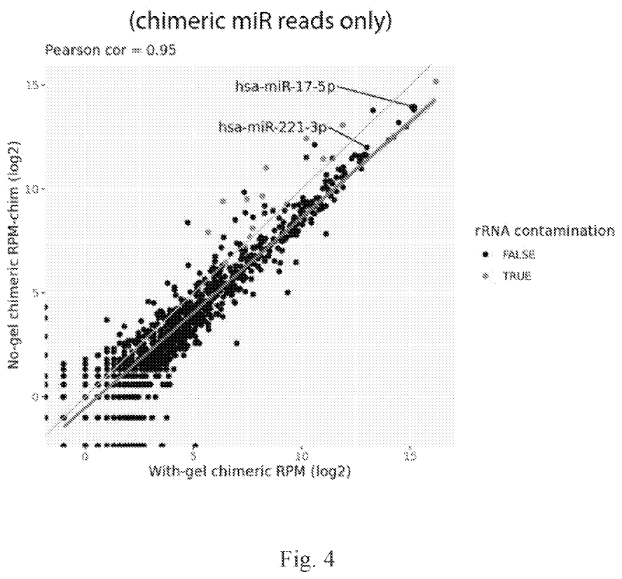

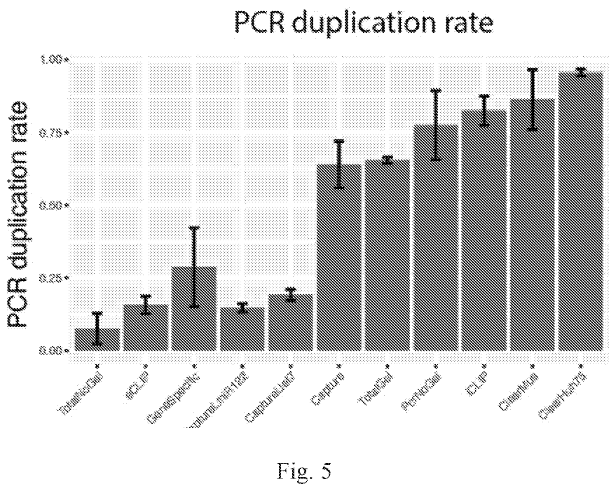

miR-eCLIP adds a specialized chimeric ligation to AGO2 eCLIP and boosts chimeric rate more than eight-fold, it goes up from 0.3% in standard AGO2 eCLIP (includes gel step) to 2.7% in miR-eCLIP libraries with gel (FIG. 1 and FIG. 2). Chimeric rate is expressed as a ratio of PCR deduplicated uniquely mapped chimeric reads and a sum of counts of deduplicated uniquely mapped chimeric and non-chimeric reads. As expected, skipping the gel step resulted in overall lower chimeric rate (1.1% in HEK293×T cells) (FIG. 2). FIG. 2 shows that chimeric rate in Total Chimeric miR-eCLIP no-gel assay is in between AGO2 eCLIP and Total Chimeric miR-eCLIP with-gel. However, since omission of the gel clean up step greatly simplifies the workflow making it suitable for high throughput automation, and since the gel omission did not result in a strong bias in IP enrichment or distribution of chimeric reads (FIG. 3 and FIG. 4). It was reasoned that no-gel miR-eCLIP is suitable as a platform to develop miR-eCLIP with an added chimeric read enrichment step. Chimeric rate in miR-eCLIP libraries that were enriched for chimeras specific to one or more miRNAs of interest using probe capture was much higher, ranging from 7% in HEK293×T libraries to almost 30% in mouse liver. It should be noted that even though total and probe capture enriched miR-eCLIP chimeric libraries were sequenced at least three-fold deeper than CLEAR-CLIP libraries, miR-eCLIP libraries with or without enrichment still had a greater complexity resulting in 20% lower PCR duplication rate (FIG. 5). FIG. 5 shows greater PCR duplication rate in the external datasets relative to miR-eCLIP and eCLIP. miR-eCLIP and AGO2 eCLIP experiments performed in HEK293×T cells, unless labelled otherwise.

miR-eCLIP Recovers miRNA:mRNA Chimeras

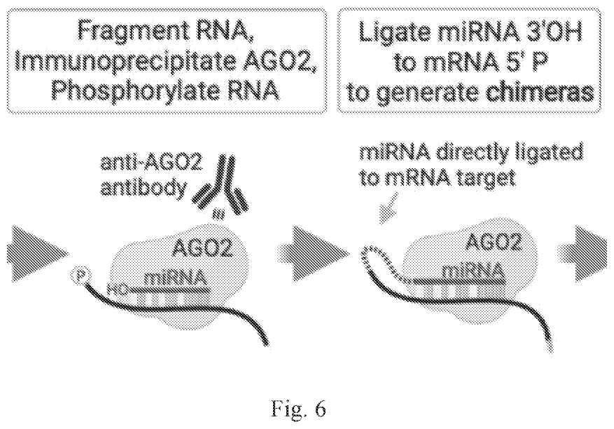

Chimeric CLIP-seq approaches (including CLASH, CLEAR-CLIP, and other chimeric CLIP-seq approaches have shown that chimeric ligation of miRNAs to their mRNA targets is encouraged by the addition of a ligation step (without adapters) to encourage proximity-based ligation. Thus, it was desired to set out to build upon the improved library preparation steps in the enhanced CLIP (eCLIP) procedure was developed by incorporating this chimeric ligation step. It was observed that the dephosphorylation steps in standard eCLIP would inhibit chimera generation by removing terminal 5′ phosphates from the mRNA fragments generated by limiting RNase treatment. Therefore, an additional phosphorylation step (using 3′ phosphatase minus T4 Polynucleotide Kinase (NEB)) and an additional ligation step to convert eCLIP to chimeric eCLIP was implemented (FIG. 6). Additionally, the size selection steps were modified by using less ethanol during beads cleanup, which selectively reduced binding of shorter fragments and enriched for fragments of at least 40 nt and reduced miRNA-only reads.

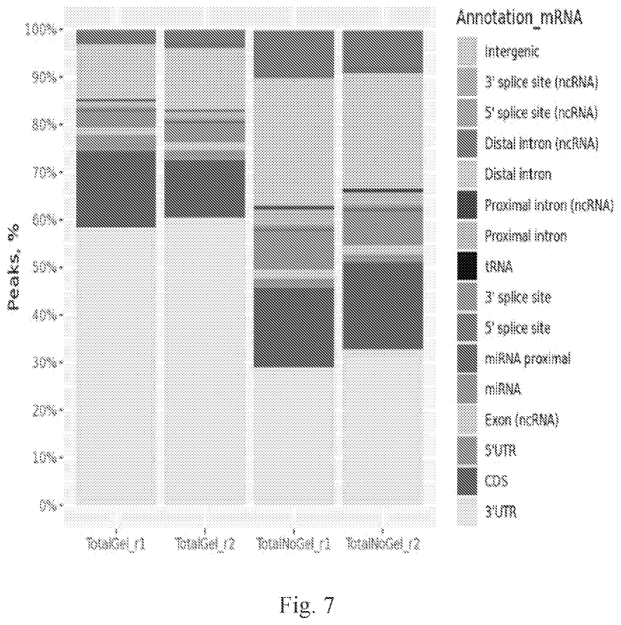

To test whether this approach successfully recovers microRNA targets, chimeric eCLIP on HEK293T cells using a previously validated AGO2 antibody and a standard eCLIP library prep was performed, which includes polyacrylamide gel step. Two libraries that were sequenced with 144 and 145 million reads each were generated. As the majority of reads lack chimeras, standard CLIP analysis, including adapter trimming, repetitive element removal, genomic mapping, PCR duplicate removal, and peak calling was performed first. Confirming that the AGO2 interactions was successfully enriched, it was observed that 59.4% of peaks were located in 3′UTRs (with another 14.5% in coding sequence (CDS)) (FIG. 7). FIG. 7 shows about 2-fold greater frequency of intronic, lincRNA and miRNA peaks in no-gel miR-eCLIP libraries relative to with-gel miR-eCLIP. In summary, these results indicate successful enrichment of both miRNAs and putative targets in 3′UTR and CDS regions with AGO2 eCLIP.

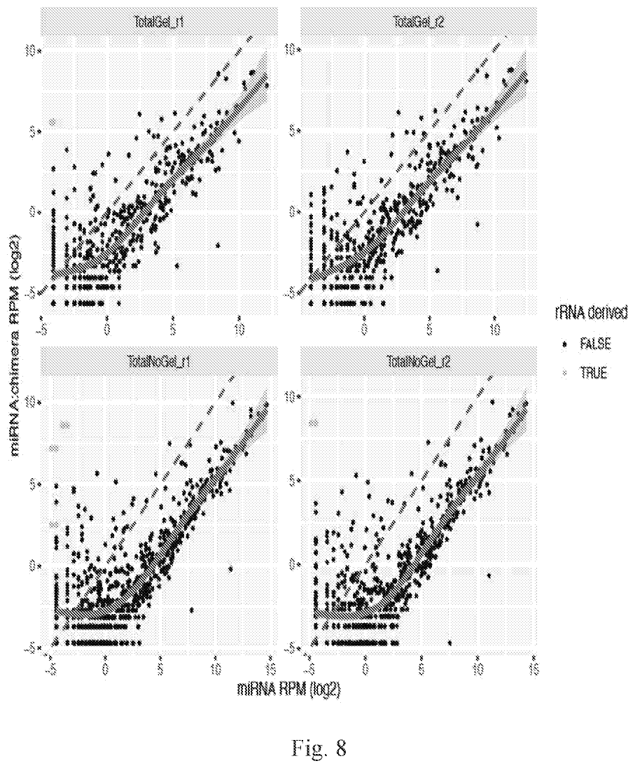

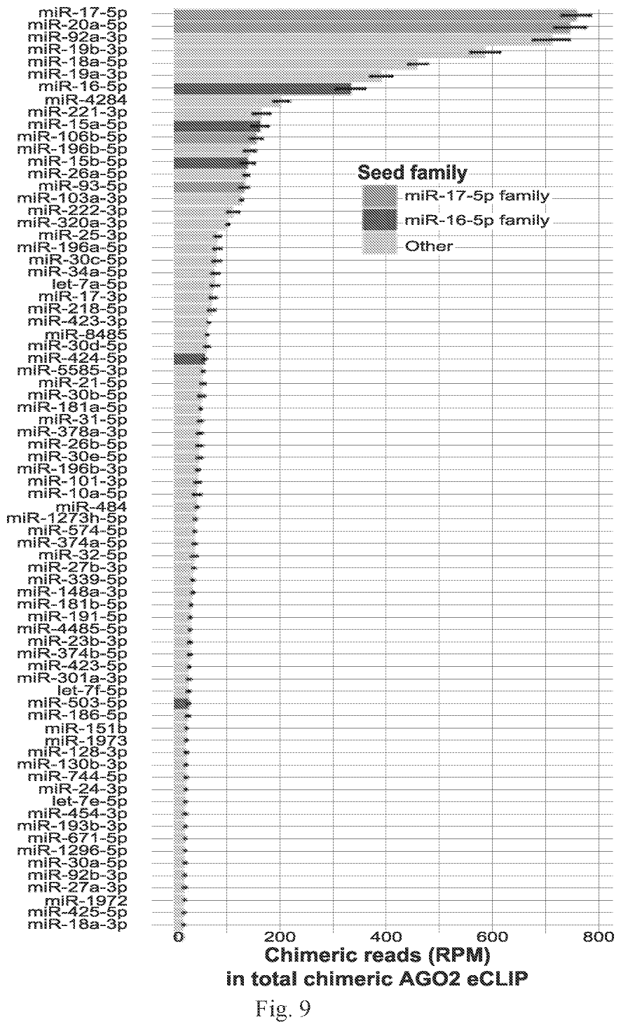

Next, chimeric reads in these libraries were considered, using a modified pipeline based on a previously published ‘reverse mapping’ strategy. Two replicate with-gel total chimeric-eCLIP libraries prepared from HEK293×T cells contained a total of 451 k and 479 k unique chimeric reads (0.3% of 145M initial sequenced reads per library, or 2.7% of uniquely mapped deduplicated reads) (FIG. 1). FIG. 1 shows that chimeric rate is greater with added chimeric ligation than without (AGO2 eCLIP v. miR-cCLIP and CLEAR CLIP) and that chimeric rate with Probe Enrichment is greater than with No Enrichment. Chimeric rate is expressed as a ratio of PCR deduplicated uniquely mapped chimeric reads and a sum of counts of deduplicated uniquely mapped chimeric and non-chimeric reads. Error bars show standard deviation. As expected, it was observed high correlation between miRNA-only and miRNA:chimera reads (FIG. 8). With Gel miR-cCLIP samples miR-eCLIP (TotalGel r1, TotalGel r2), and two Total Chimeric No Gel miR-eCLIP samples (TotalNoGel r1, TotalNoGel r2) depiciting a high correlation between miRNA-only and miRNA:chimera reads (Pearson Correlation greater than 0.68 for RPM of non-chimeric reads was computed as a sum of uniquely mapped non-chimeric reads (deduplicated) and multimapped non-chimeric reads (also deduplicated) divided by the total number of mapped deduplicated reads (both uniquely mapped and multimapping) times IM RPM of chimeric reads was calculated as a number of chimeric reads divided by the sum of mapped non-chimeric reads (deduplicated) and chimeric reads (deduplicated) times 1M. The dashed line shows identity. The curved line shows loess fit with 0.95 confidence intervals. Nineteen percent of chimeric reads were removed from further analysis because they corresponded to likely erroneously annotated miRNAs with sequences that can be mapped to rRNA (list of 15 filtered miRNA IDs. After filtration, these experiments yielded 5,000-20,000 chimeric reads per miRNA for the top 10 identified miRNAs, rapidly declining to less than 1000 chimeric reads for the 50th most abundant miRNA (FIG. 9). FIG. 9 shows that chimeric abundance is relatively high for top 10-15 miRNAs, but it peters down beyond that. Error bars show standard deviation, n=2. The top-75 list of miRNAs is defined by number of chimeric reads identified for each miRNA. RPM is calculated as number of chimeric reads divided by the sum of mapped non-chimeric reads (deduplicated) and chimeric reads (deduplicated) times 1M. miRNAs belonging to two seed families abundant in HEK293×T cells are highlighted. Interestingly, miRNAs from two miRNA seed-families (miR-17-5p and miR-16-5p families) were overrepresented among top miRNAs, which implies that chimeric rate similarity can be indicative of a similar miRNA function.

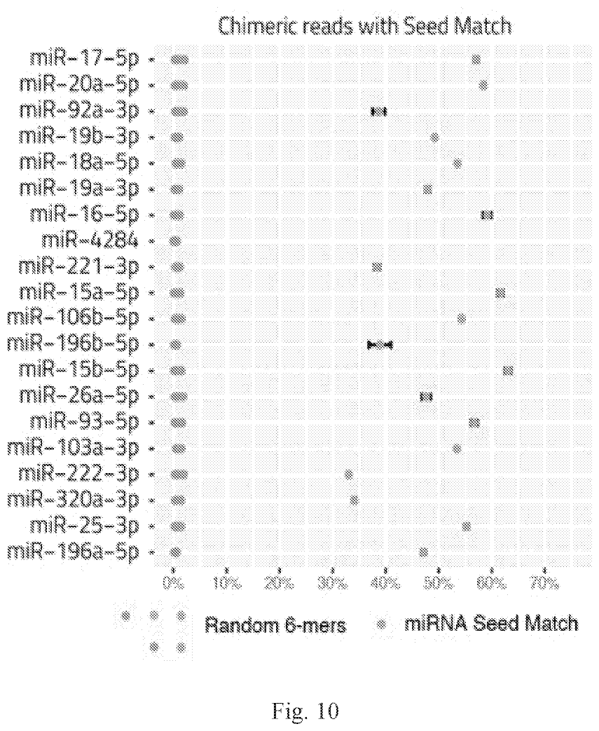

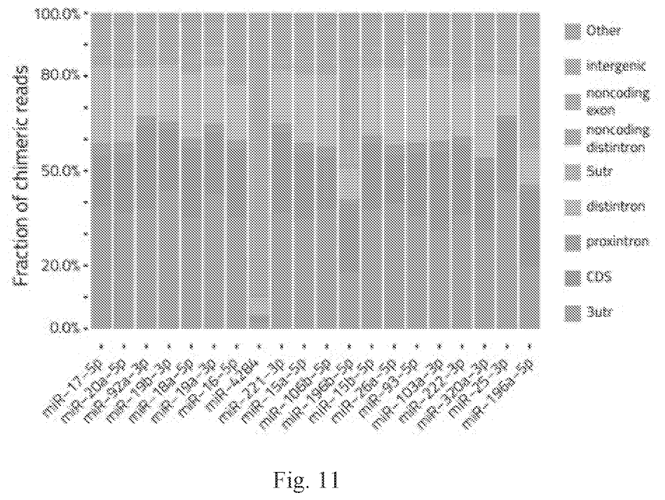

To confirm whether the chimeric reads likely reflected true miRNA targets, a variety of properties were considered. First, sequence analysis showed that for all but one miRNA among the top 20, there was 30 to 100-fold enrichment for presence of the cognate 6-mer seed matching site in the target portions of chimeric reads relative to background, with a large percentage of chimeric reads (30%-62%, depending on miRNA) containing the seed matching site (FIG. 10). FIG. 10 shows that the seed matching sites are significantly enriched in target portions of chimeras of top-20 miRNAs (except miR-4284). Seed match is defined as sequence reverse complementing positions [2:7] of mature miRNA sequence (from miRBase). Frequency of occurrence of 5 random 6-mers is shown for comparison providing a way to empirically estimate p.value of seed matching site enrichment: if frequency of the seed match occurrence is greater than that of all 5 random 6-mers, then the empirical p.value is less than 5/100=0.05. Random 6-mers were sampled from the multinomial distribution of single nucleotides occurrences (A, C, G and T), where frequency of occurrence of each nucleotide was estimated from target portions of with-gel Total Chimeric reads. Random 6-mers were controlled to not match seed regions of any human miRNA annotated in miRBase. The background frequency was approximated as frequency of occurrence of 5 random 6-mers that were sampled from the multinomial distribution of single nucleotides occurrences (A, C, G and T), where frequency of occurrence of each nucleotide was estimated from target portions of chimeric reads. Random 6-mers were controlled to not match seed regions of any human miRNA annotated in miRBase. Frequency of the random 6-mers provides a way to empirically estimate p-value of seed matching site enrichment relative to the background: if frequency of the seed match occurrence is greater than that of all 5 random 6-mers, then the empirical p-value is less than 5/100=0.05. One exception to this rule was miR-4284, which was found to be predominantly associated with transcripts of mitochondrial origin without a seed match. Next, location analysis again indicated an enrichment for expected target regions. With the exception of miR-4284, 33% of chimeric reads mapped to 3′-UTRs and additional 19% to CDS (FIG. 11). FIG. 11 shows that over 50% of chimeric reads for most miRNAs in top-20 (by chimeric read count) are mapped to 3′UTR and CDS regions. Fractions (expressed as percent, the y-axis) of each partition is a ratio of the mean count (n=2) of chimeric reads of each miRNA mapped to each partition divided by the mean of chimeric reads per miRNA. These results indicate that the chimeric reads obtained with chimeric eCLIP modifications have properties that match previous chimeric CLIP-seq approaches.

Validation of No-Gel Chimeric eCLIP for miRNA Target Profiling

The standard eCLIP protocol that chimeric eCLIP is based includes SDS-PAGE protein gel electrophoresis, Western blot-like nitrocellulose membrane transfer, and manual cutting of the membrane to isolate protein-crosslinked RNA. These steps are performed for two purposes: first, non-crosslinked RNA does not transfer to nitrocellulose and is thus removed, and second, denaturation removes co-immunoprecipitated unwanted proteins of different size than the targeted protein. However, in addition to being complex for novice users and limiting scalability and automated handling, it was observed that this transfer and isolation step by itself drives a dramatic reduction in experimental yield. As experience with other RBPs suggested that co-immunoprecipitation artifacts were heavily protein- and antibody-dependent, it was thus tested whether removing these steps altered composition of chimeric cCLIP-reads.

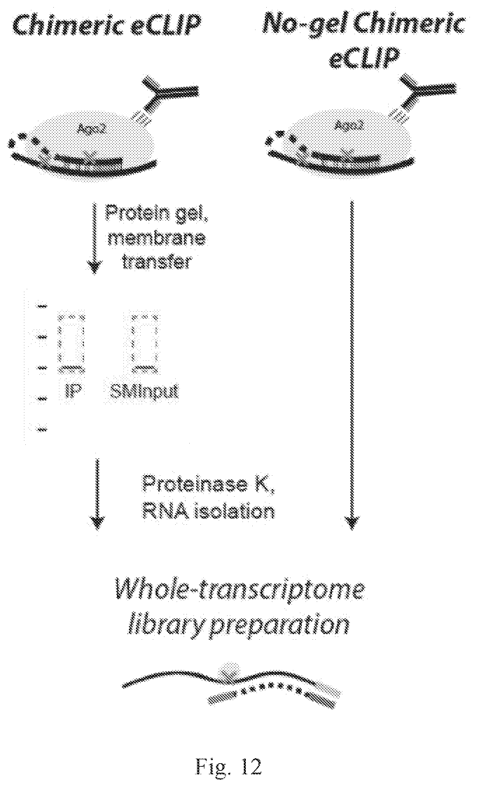

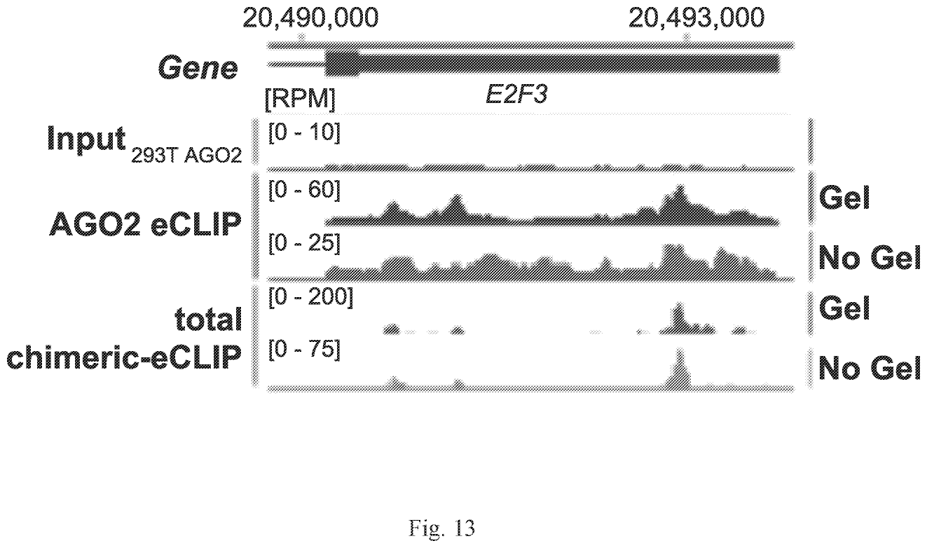

To do this, side-by-side testing with a simplified protocol was performed that removes the SDS-PAGE and membrane transfer steps and replaces it with a simple Proteinase K treatment to isolate the crosslinked RNA (“no-gel” variant of chimeric eCLIP (FIG. 12). It was observed that removal of the gel transfer steps required on average ˜6.5 fewer PCR cycles of amplification, suggesting ˜100-fold increased experimental yield. Manual inspection suggested similar read density distributions of non-chimeric as well as chimeric cCLIP reads between with-gel and no-gel libraries (FIG. 13). To explore this further, with-gel and no-gel enrichment of non-chimeric IP reads were compared relative to size matched input libraries across 375 k regions identified as clusters of reads in the with-gel IP libraries by CLIPper. It was observed that the no-gel approach resulted in a greater enrichment of IP libraries over miRNA genes, indicating that miRNA-only reads make a greater contribution to the non-chimeric reads in the no-gel libraries (FIG. 7). Despite higher contribution of miRNA-only reads, the overall pattern of non-chimeric read density distribution and IP enrichment was well preserved in the no-gel libraries. IP/input ratio was strongly correlated between with-gel and no-gel libraries transcriptome wide (Pearson correlation 0.82, P·Value<2.2·10⁻¹⁶ (FIG. 3). FIG. 3 shows overall strong correlation (0.82) of IP/input enrichments values from no-gel and with-gel experiments. FIG. 3 also reveals a plume of clusters, enriched for those over miRNA-genes (the light grey points), that have greater IP/input enrichment (i.e. ratio of RPMs) in the no-gel assay relative to with-gel. Clusters plotted on this figure were identified in replicate 1 of Total Chimeric miR-eCLIP with-gel experiments. The top line shows identity, the bottom line shows least squares linear model fit. The 95% confidence intervals around the fit are also shown, but the range is too tight to be seen on the plot.

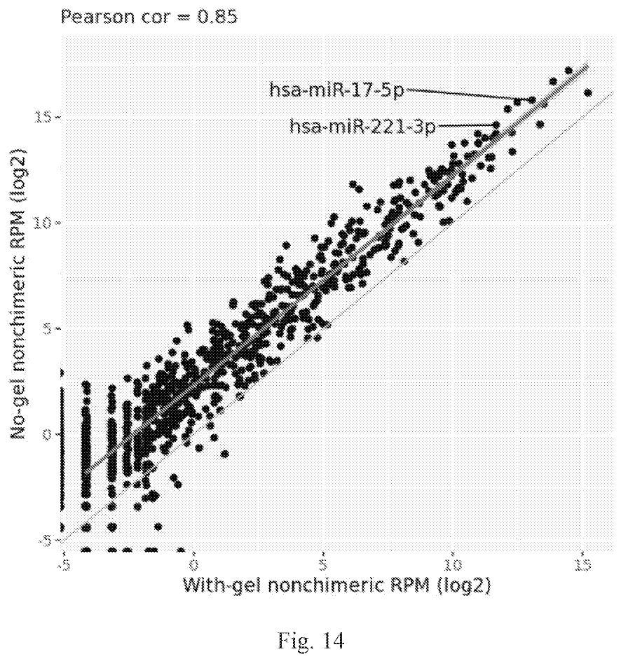

Composition of miRNAs in non-chimeric reads was also well preserved between with-gel an no-gel approaches, resulting in a high correlation of miRNA read counts between the methods (Pearson correlation 0.85, P·Value<2.2·10⁻¹⁶) (FIG. 14). Correlation of non-chimeric and chimeric read counts of miRNAs was similarly high between with- and no-gel variants (Pearson correlation 0.73 with-gel variant, and 0.85 in the no-gel variant, P·Value<2.2·10⁻¹⁶ (FIG. 8). Finally, high correlation of miRNA-chimeric read counts between with-gel and the no-gel versions of the assay (Pearson correlation 0.95, P·Value<2.2·10⁻¹⁶) shows that relative composition of chimeric reds was faithfully preserved in the no-gel approach (FIG. 4). FIG. 4 shows overall strong correlation (0.95) between no-gel and with-gel assays. The top line shows identity, the bottom line shows least squares linear model fit along with 95% confidence intervals. Values on x-axis are a mean of RPMs of two replicates of with-gel Total Chimeric miR-cCLIP experiments, y-axis is a mean of RPMs of two replicates of no-gel experiments. RPMs here were calculate off chimeric reads only, i.e. RPM was defined as a ratio of chimeric reads per-miRNA and the total number of chimeric reads in the library.

These and further validations described below indicated that the no-gel chimeric eCLIP variant did not introduce a substantial bias among chimeric reads and is well suited as an easy-to-use unbiased platform for developing chimeric enrichment approaches.

### Targeted Enrichment by Probe-Based Capture

To address these concerns, a probe-capture enrichment technique with modified oligonucleotides to increase the depth of chimeric read enrichment was tested. Probe-capture chimeric-eCLIP can enrich for entire miRNA families while preserving the exact sequence of the specific miRNA bound to each target mRNA, enabling deep profiling of miRNA families with highly overlapping sequences. Furthermore, it allows for exact identification of the 5′-end of the miRNA from chimeric reads, which has proven insightful in understanding the role untemplated 5′ nucleotides play in modulating miRNA targeting.

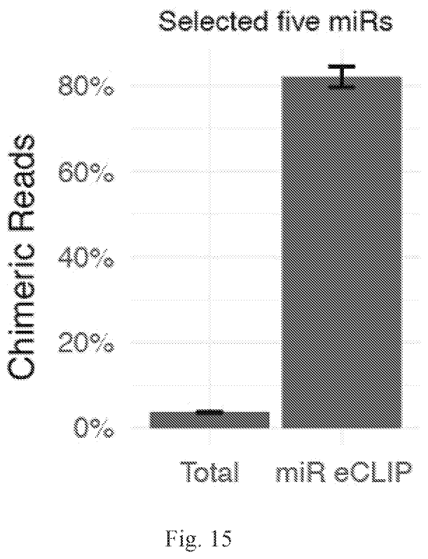

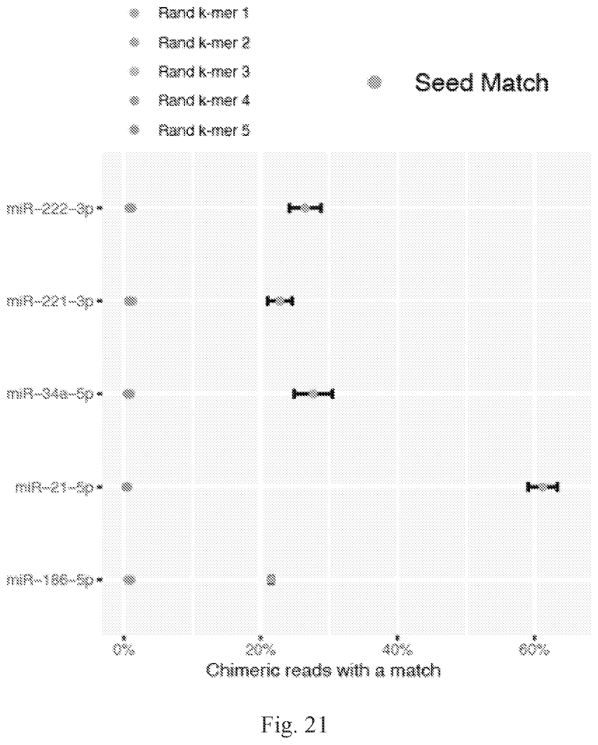

First, specificity of enrichment of chimeric reads for miRNAs of interest in a cell line was tested. miR-eCLIP to enrich libraries for chimeras of five miRNAs of interest in HEK293×T cells (miR-221-3p, miR-34a-5p, miR-186-5p, miR-21-5p and miR-222-3p) was applied and compared it to libraries generated using miR-eCLIP without enrichment (total chimeric libraries) (FIG. 15). These miRNAs were chosen to span a range of miRNA abundances, with the most abundant miRNA out of the five ranked top-14 most highly expressed miRNA, while the least abundant ranked top-56 most highly expressed miRNA in HEK293×T (according to smallRNA-seq profiling). Overall proportion of chimeric reads in the enriched libraries was 2.5-fold higher than in libraries prepared without enrichment (FIG. 1), while among the chimeric reads contribution of reads for the targeted miRNAs increased more than 20-fold (FIG. 21). In summary, the results show that, with respect to initial libraries reads, the yield of chimeric reads for enriched miRNAs increased more 50-fold in probe capture miR-eCLIP libraries relative to total (non-targeted) miR-eCLIP. Of note, it was observed over 50,000 chimeric reads for each of enriched miRNAs; this depth would typically require multiple full sequencing flow cells with traditional chimeric CLIP-seq approaches.

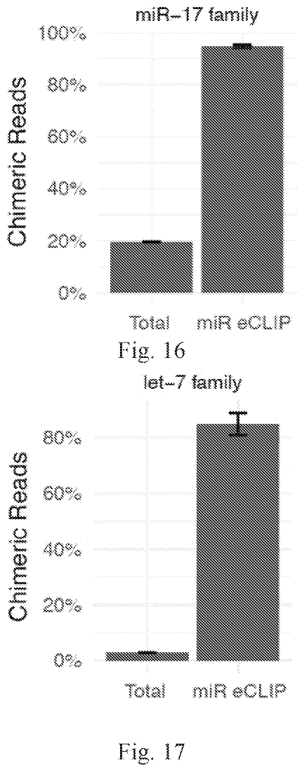

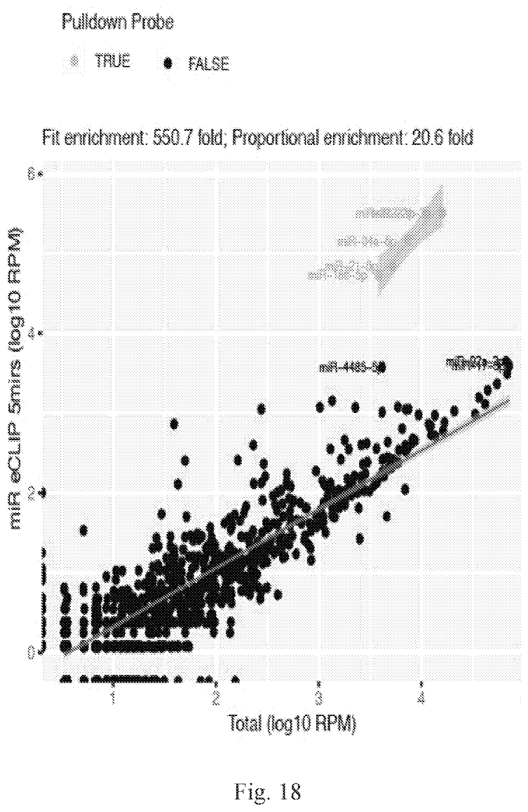

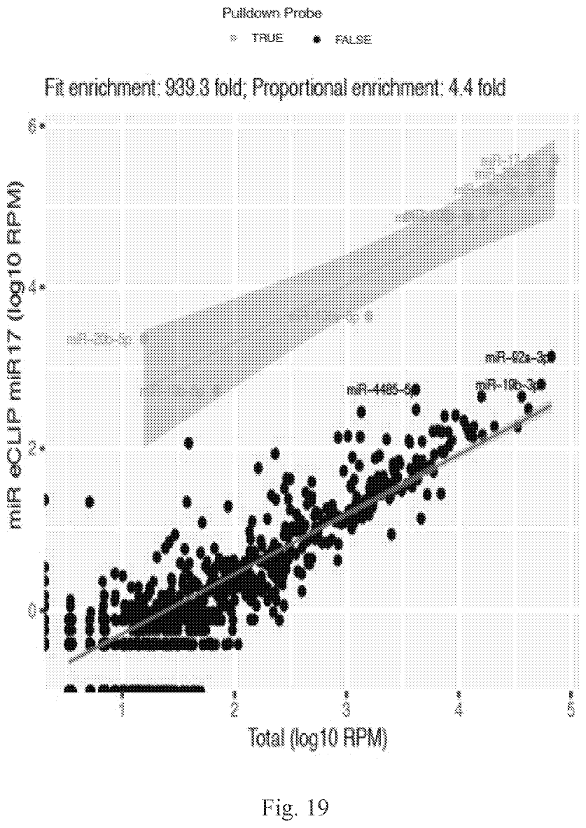

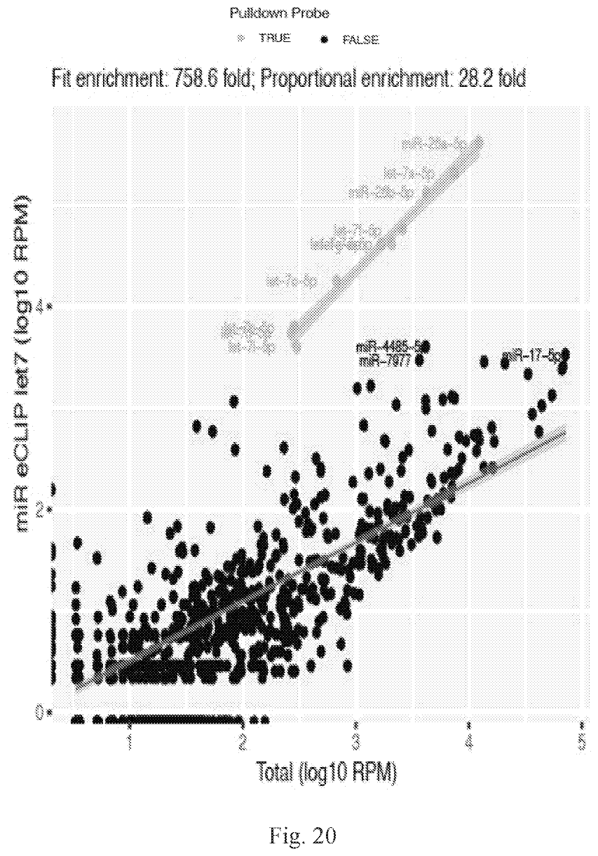

Furthermore, since many investigators are interested in studying families of miRNAs, probe capture were tested to see if they could simultaneously and specifically enrich chimeric reads for members of the same miRNA family, even if family members have very different miRNA abundances. It was chosen to target six members of miR-17 family (miR-17-5p, miR-93-5p, miR-20a-5p, miR-20b-5p, miR-106a-5p, miR-106b-5p) along with two miRNAs with related seed sites (miR-18a-5p, miR-18b-5p). miR-17 family includes two highly expressed miRNAs in HEK293×T (2^{nd} most abundant miR-20a-5p, and 5th most abundant miR-93-5p), while three miRNAs (miR-20b-5p, miR-106a-5p and miR-18b-5p) are ranked outside of top-200 most abundant miRNAs (FIG. 17). Two members of let-7 family (let-7a-5p and let-7g-5p) were profiled along with two miRNAs of interest that were unrelated to let-7 family (miR-26a-5p and miR-26b-5p). Members of let-7 miRNA family are highly similar to each other and as expected, use of probes for let-7a-5p and let-7g-5p resulted in enrichment of other members of let-7 family (let-7b-5p, let-7c-5p, let-7d-5p, let-7e-5p, let-7f-5p and let-7i-5p) (FIG. 20). miRNAs in let-7 family experiment also varied in abundance (lowest expressed miRNA was let-7d-5p ranked top-135, and highest expressed was let-7a-5p, ranked top-8). The preliminary results showed that in a single experiment, probe capture can simultaneously enrich for multiple miRNAs as compared to chimeric read population of the total (not enriched) libraries. Even though miRNAs that were selected in the miR-17 family experiment accounted for almost 20% of total chimeric reads without enrichment, miR-eCLIP still worked to farther increase representation of selected miRNAs resulting in over 94% of chimeric reads being accounted for by the selected miRNAs after enrichment (4.4-fold increase, (FIG. 16)). Chimeric reads for miRNAs in the let-7 family experiment were less common, accounting for less than 3% of total chimeric reads without the enrichment. In miR-eCLIP library, proportion of chimeric reads for the selected miRNAs have increased to over 84% (28-fold increase, (FIG. 17)). Analysis of distribution of chimeric reads among individual miRNAs shows that in each experiment the increase in read counts was specific to miRNAs intended to be enriched by probe design (FIG. 18, FIG. 19, and FIG. 20).

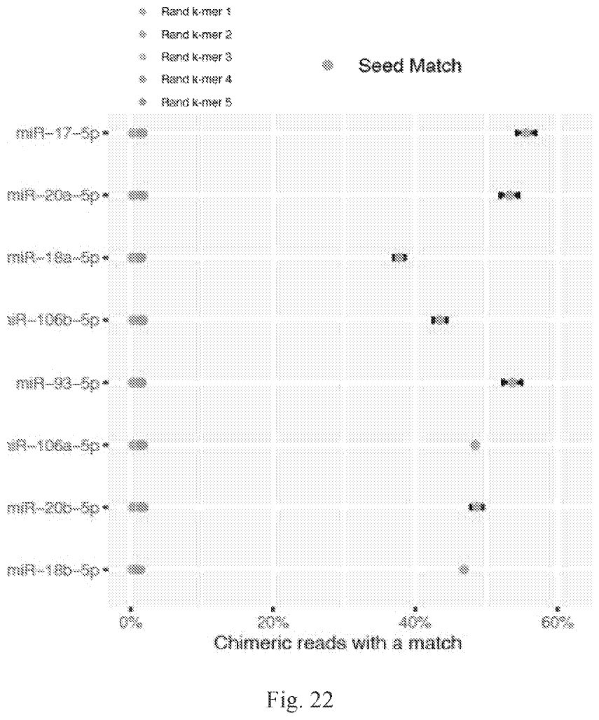

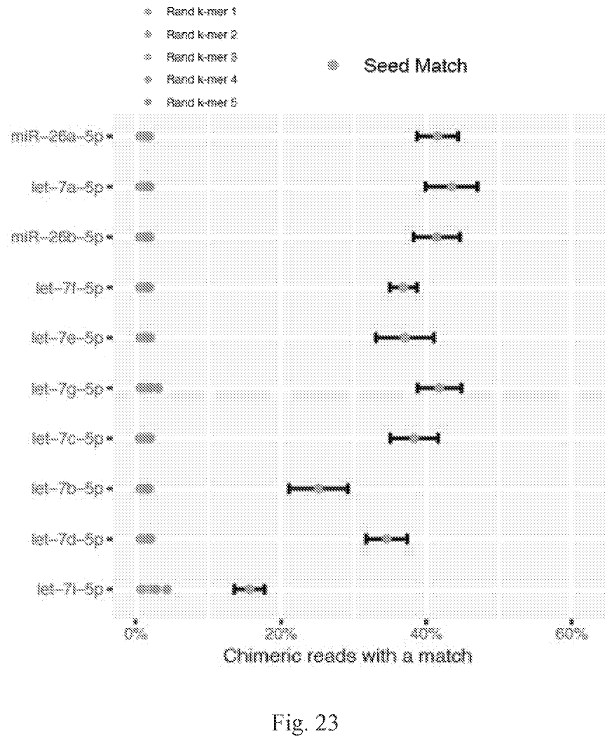

It was found that in the target portions of chimeric reads, 6-mers complementary to [2:8]-seed sequence of the cognate miRNAs occur over 35-times more commonly than expected from background frequency of single nucleotides alone. This result matches a biological expectation given role of seed complementarity in target recognition and stabilizing of AGO2 binding to target transcript. The proportion of reads with seed matches to cognate miRNAs varies between different miRNAs reaching over 50% for three miRNAs in miR-17 family (FIG. 21, FIG. 22, and FIG. 23).

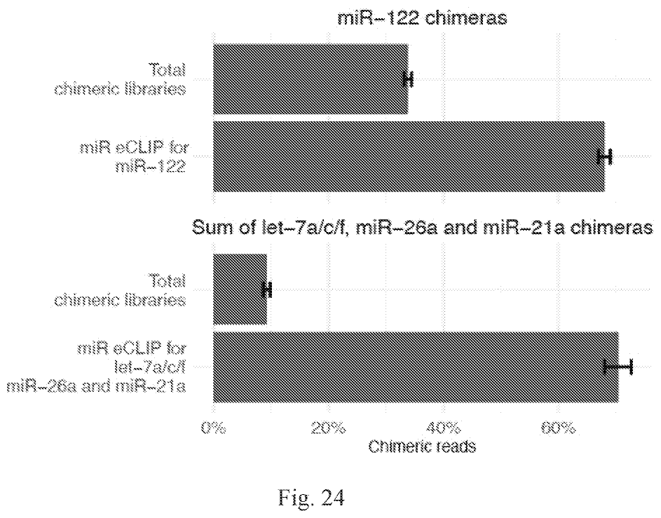

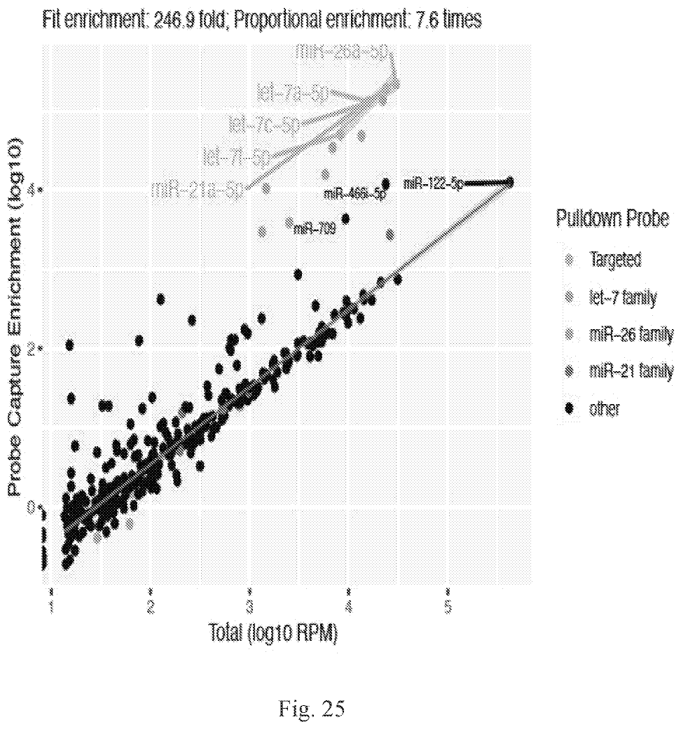

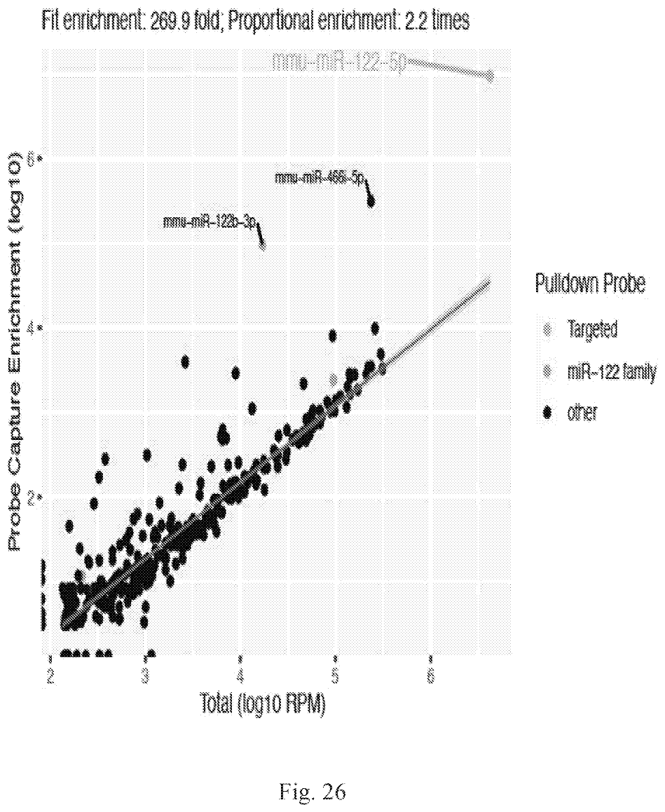

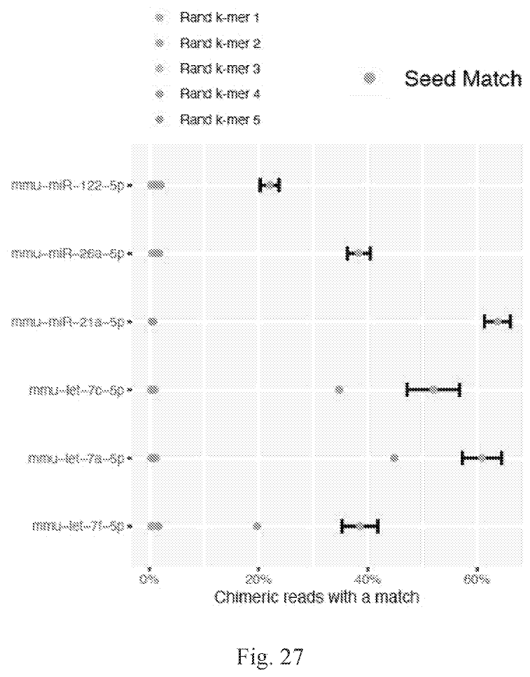

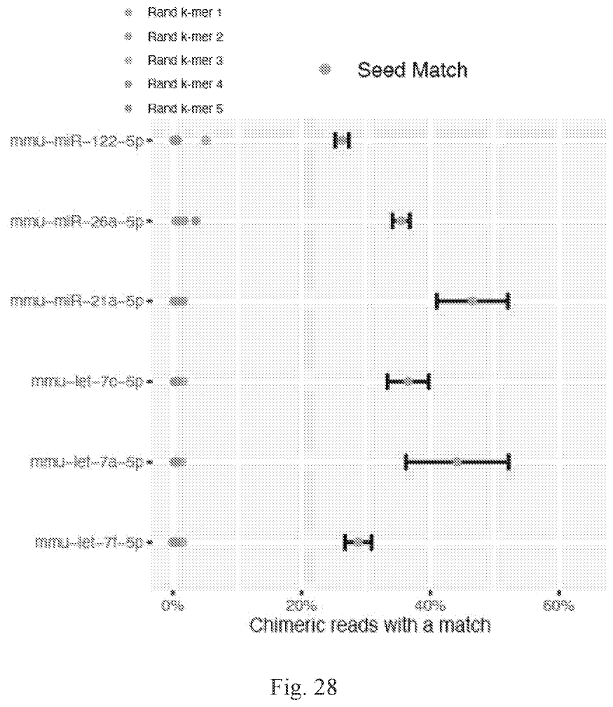

Finally, accuracy and efficiency of miR-eCLIP enrichment of miRNA:mRNA chimeras were tested in a different kind of a clinically relevant sample time, a mouse liver tissue. Enriched libraries were compared to standard AGO2 eCLIP libraries with an added chimeric ligation step prepared from the same tissue samples. Two sets of enriched libraries were prepared, one was enriched for a selection of five miRNAs (miR-26a-5p, miR-21a-5p, let-7a-5p, let-7c-5p, let-7f-5p) and another was enrichment specifically for miR-122-5p. Chimeric rate, expressed as a ratio of chimeric reads and all uniquely mapped reads, was at least 4 to 6-fold higher in liver miR-eCLIP libraries than with previously published methods, resulting in 20% and 30% chimeric rate in libraries enriched for miR-122-5p and a set of five miRNAs, respectively (FIG. 1). At the same time, the enrichment also increased representation of chimeras for miRNAs of interest among chimeric reads (FIG. 24). In the experiments with five selected miRNAs, proportion of chimeric reads for miRNAs of interest had increased from 9% in total chimeric library to over 70% in miR-eCLIP libraries (7.5-fold increase). Representation of chimers of a highly abundant miR-122-5p miRNA has also increased. Without enrichment, miR-122-5p chimeras account for 34% of total chimeric reads, while in miR-cCLIP enriched libraries, proportion of miR-122-5p chimeras was 68% (2.2-fold increase). Analysis of per-miRNA read counts showed that increase in chimeric read count was specific to enriched miRNAs, with only other miRNAs with substantial increase in chimeric reads being related family members (FIG. 25 and FIG. 26). As in HEK293×T libraries, seed matching sites for miRNAs in liver chimeric reads were significantly over-represented in target portions of chimeric reads relative to background, in agreement with expectations given biological role of seed complementarity in miRNA targeting (FIG. 27 and FIG. 28). In the end, starting with 40-45M of initial reads, miR-eCLIP libraries contained from hundreds of thousands to several million of chimeric reads per miRNAs of interest, which is orders of magnitude greater than was possible to achieve previously.

Deep Profiling of miRNAs Targeting Gene of Interest

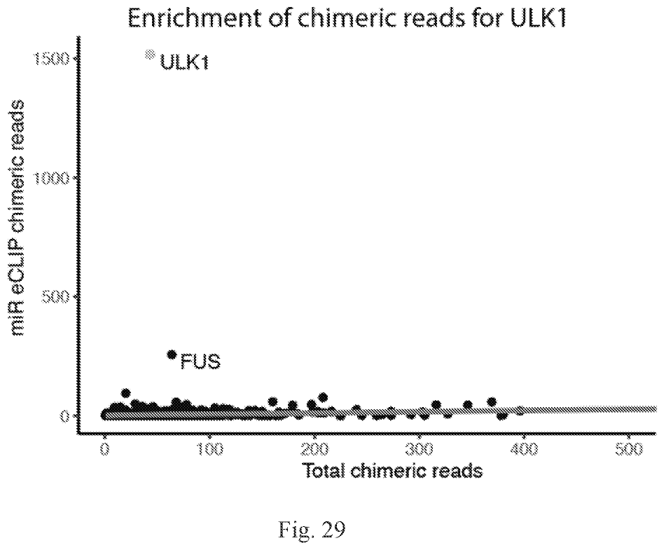

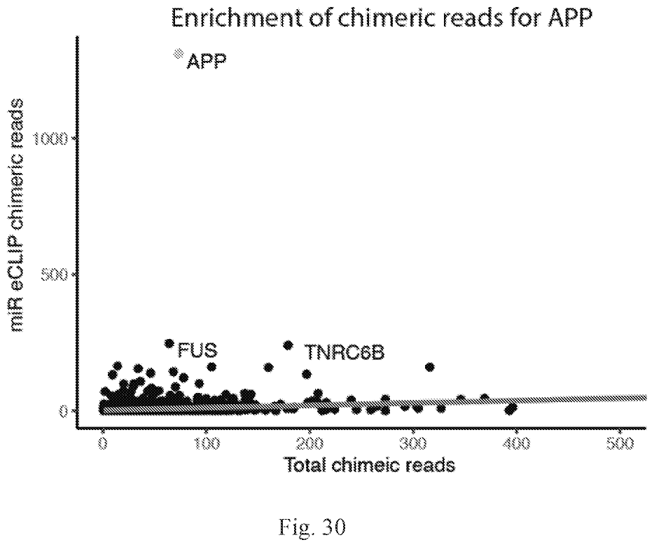

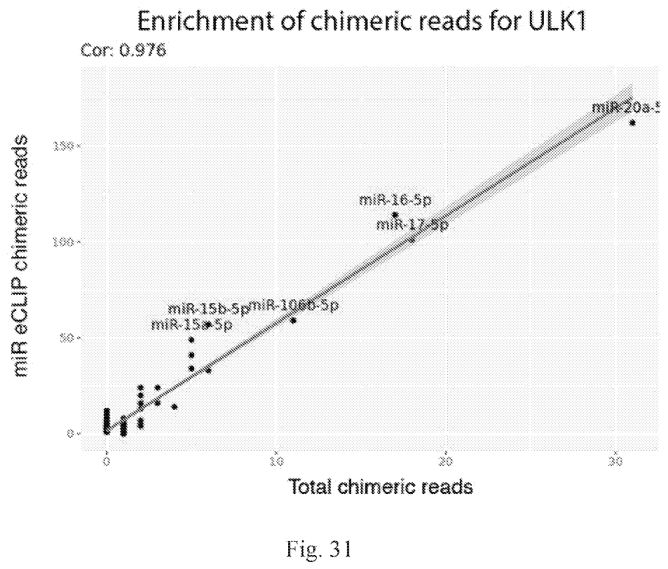

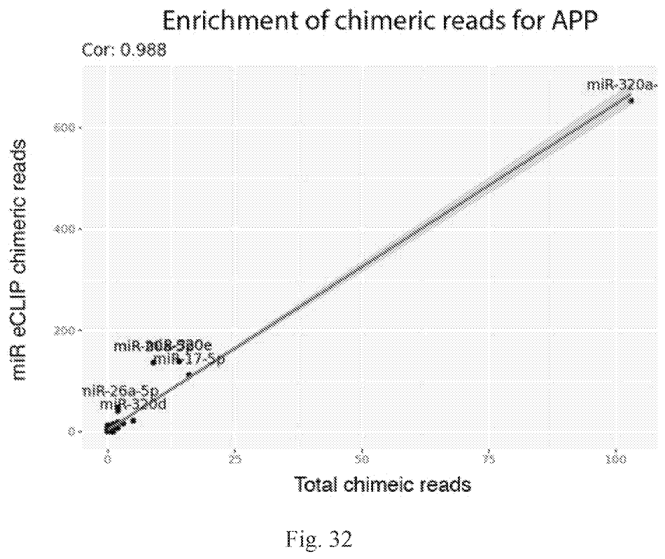

While profiling of genes targeted by individual miRNAs is important, it is also important to be able to address a reciprocal challenge of comprehensively identifying miRNAs that targeted a specific gene of interest. Application of miR-eCLIP was tested to address this question by designing enrichment probes to complement sequence of a gene of interest, rather than sequence of miRNAs of interest. Libraries enriched for gene of interest chimeric reads had overall fewer chimeric reads, but representation of chimeric reads for the gene of interest has increased 50-fold and 300-fold in APP and ULK1 enrichment experiments, respectively (FIG. 29 and FIG. 30). This resulted in identifying of over a thousand chimeric reads specific to genes of interest in each enriched library. Despite differences in chimeric read abundance with and without enrichment, counts of chimeric reads per miRNA was highly correlated between enriched libraries and matched libraries prepared using miR-eCLIP without probe enrichment (Pearson correlation >0.97, (FIGS. 31 and 32). This result confirmed that no major biases in miRNA representation among gene specific chimeras was introduced by a substitution of gel clean up step with the probe capture enrichment.

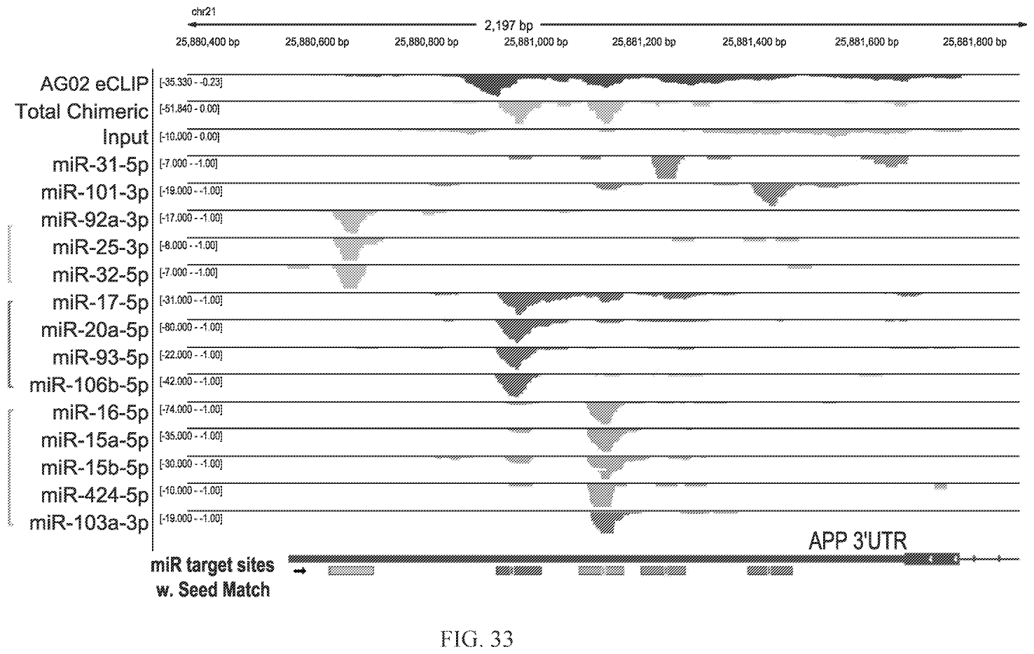

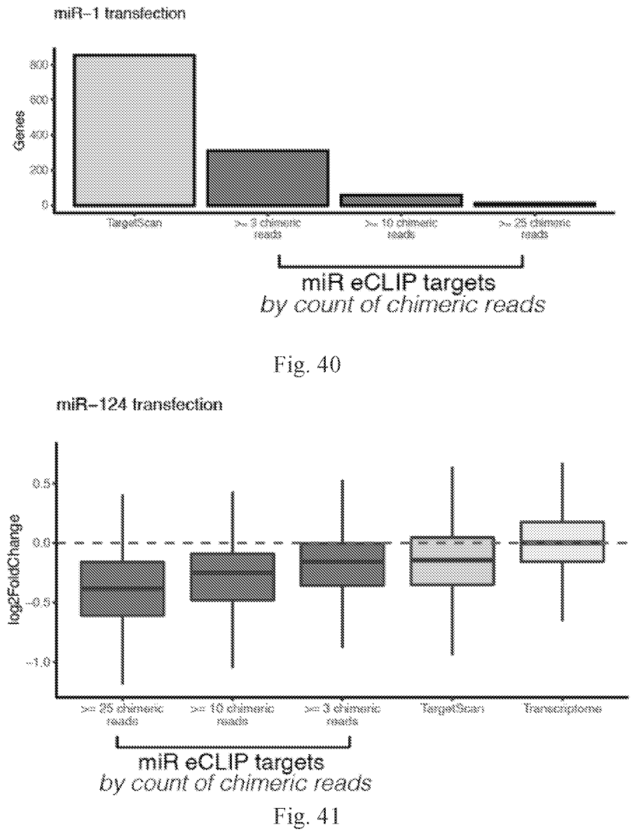

Examining chimeric reads mapped to 3′UTRs of enriched genes showed that chimeric reads profile miRNA targeting a specific gene of interest in an unprecedented detail. Individual target sites were well separated from each other, visible as distinct peaks in chimeric read density (FIG. 39 and FIG. 40), identifying four and five actively engaged miRNA target sites in 3′UTRs of ULK1 and APP transcripts, respectively. All four sites in ULK1, and four out five sites in APP contained a 6-mer complementary to the seed regions of miRNAs most highly represented in the chimeric reads mapped to each particular site. A noteworthy feature of chimeric read distribution was that most of individual sites were targeted by several different miRNAs, where most abundant miRNAs targeting the same site had the same seed site, i.e., belonged to the same seed family (FIG. 33 and FIG. 34). Therefore, miR-eCLIP showed that different miRNAs co-target the same sites in target transcripts, which helps to explain robustness of miRNA regulatory networks and its resilience to mutations of individual miRNAs in knockout experiments.

miRNA:mRNA Chimeras Quantitatively Identify Functional miRNA Targets

As microRNAs often regulate gene expression by inducing RNA degradation, a common way to validate miRNA targets at scale is to show downregulation following miRNA overexpression. Indeed, targets identified using CLASH or similar chimeric ligation approaches showed particular enrichment for functional regulation, confirming that these methods yield high-quality sets of miRNA targets. To confirm that miR-eCLIP also identifies functional miRNA targets, two individual miRNA mimics were overexpressed by transient transfection (miR-1 and miR-124, both of which endogenously expressed at low levels in HEK293×T cells, ranked 65^{th} and 265^{th} most expressed miRNAs, respectively), followed by miR-eCLIP to identify targets and mRNA-seq to assess the effect of miRNA overexpression on global gene expression.

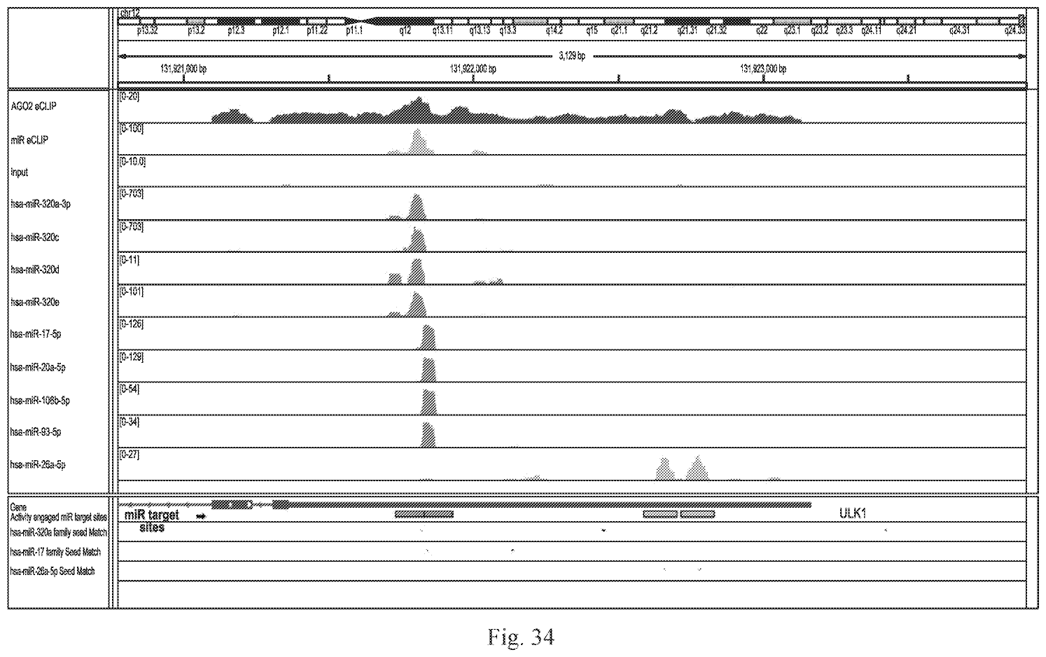

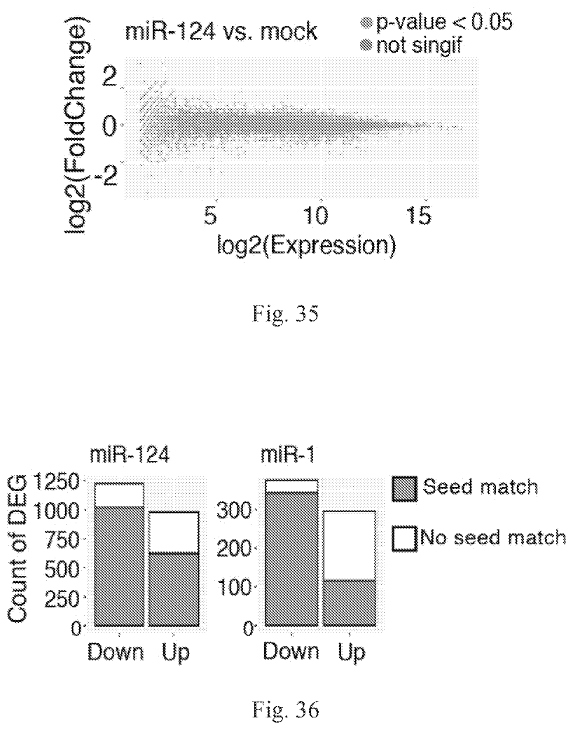

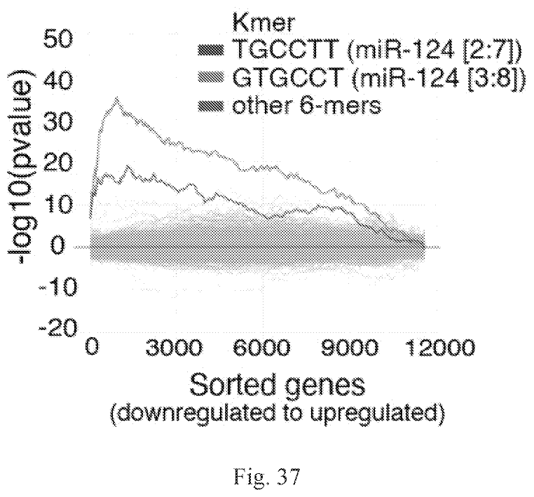

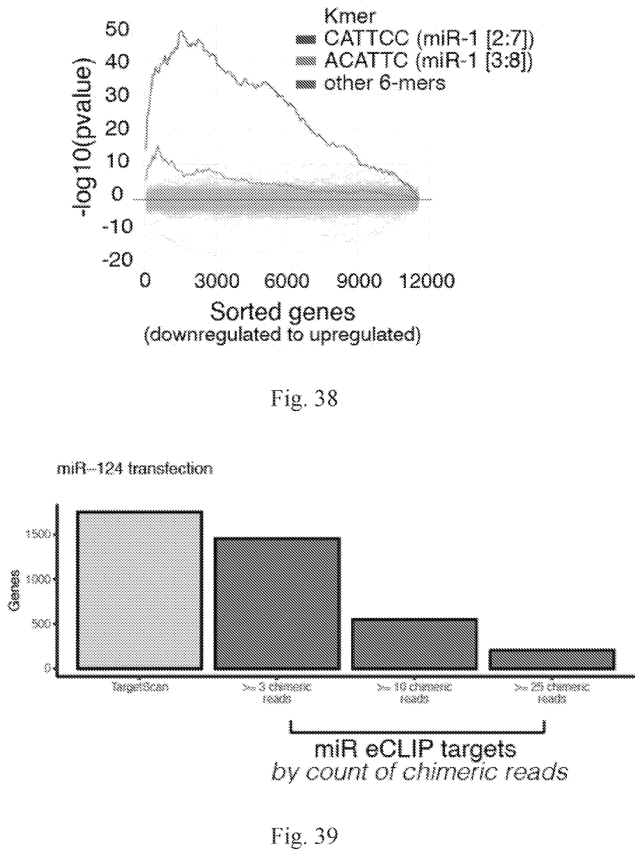

First, using DESeq2 to quantify differential gene expression were used upon miRNA overexpression (FIG. 35). As expected, 3′UTRs of downregulated contained miR-1 and miR-124 seed matches more often than 3′UTRs of upregulated genes (FIG. 36). Two 6-mers that were more highly overrepresented in 3′-UTRs of downregulated genes than any other 6-mers complemented seed sites and the offset seed sites of the two transfected miRNAs (FIG. 37 and FIG. 38). In FIG. 37, the y-axis shows negative log 10 transformed hypergeometric test p-values from tests of 6-mer enrichment in 3′UTRs of genes sorted from downregulated to upregulated (the x-axis depicts sorted genes). The enrichment of 6-mers corresponding to miR-124 seed matching site are shown with top and bottom lines. All other possible 6-mers are show (grey lines) demonstrating that enrichment of 6-mers for miR-124 in 3′UTRs of downregulated genes is highly specific. In FIG. 38, The y-axis shows negative log 10 transformed hypergeometric test p-values from tests of 6-mer enrichment in 3′UTRs of genes sorted from downregulated to upregulated (the x-axis depicts sorted genes). The enrichment of 6-mers corresponding to miR-1 seed matching site are shown with top and bottom lines. All other possible 6-mers are show (grey lines) demonstrating that enrichment of 6-mers for miR-1 in 3′UTRs of downregulated genes is highly specific. This analysis confirms that transfection of miR-1 and miR-124 mimics had specifically induced repression of miR-1 and miR-124 targets, respectively.

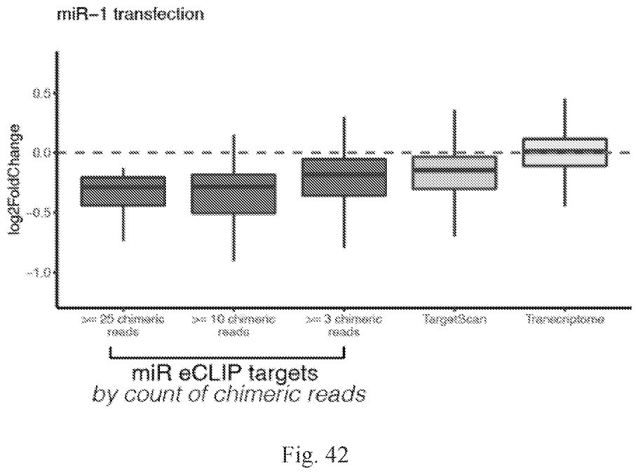

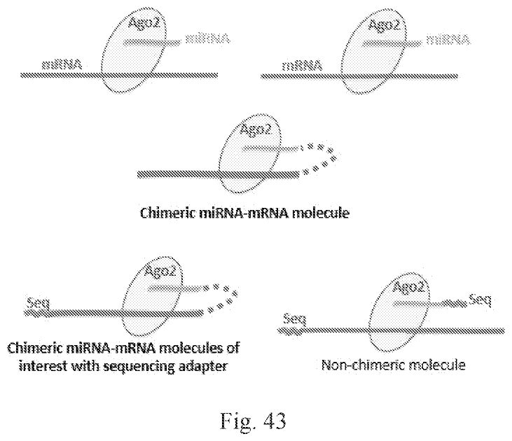

Next, miR-eCLIP were applied to identify targets of miR-124 and miR-1. To define reproducible targets, peaks using miR-124 and miR-1 chimeric reads were first called in each of the two replicates. Targets were then defined as genes with 3′-UTRs containing such chimeric peaks in both biological replicates, resulting in identification of hundreds of high confidence miRNA targets (FIG. 39 and FIG. 40). Confirming accuracy of these targets, it was observed that >75% of miR-eCLIP targets were down-regulated upon miRNA over-expression (FIG. 41 and FIG. 42). In FIG. 41, the y-axis shows differential expression of genes measured using RNA-seq that was performed in parallel with miR-eCLIP experiments to identify miR-124 targets in the transfected cells. This experiment validates miR-eCLIP targets (dark grey boxes) as functional targets, because they are repressed upon miR-124 transfection. Moreover, a set of miR-eCLIP targets is more highly enriched for functional targets than purely computational miRNA target predictions (TargetScan target predictions, shown with the light grey box, are not showing as strong of a repression as miR-cCLIP targets). Unlike computational predictions, miR-eCLIP identifies targets quantitatively: the greater the number of chimeric reads per target (labelled on the x-axis underneath the blue boxes) the deeper is target repression upon miR-124 transfection. In FIG. 42, the y-axis shows differential expression of genes measured using RNA-seq that was performed in parallel with miR-eCLIP experiments to identify miR-1 targets in the transfected cells. This experiment validates miR-eCLIP targets (dark grey boxes) as functional targets, because they are repressed upon miR-1 transfection. Moreover, a set of miR-eCLIP targets is more highly enriched for functional targets than purely computational miRNA target predictions (TargetScan target predictions, shown with the light grey box, are not showing as strong of a repression as miR-cCLIP targets). Unlike computational predictions, miR-eCLIP identifies targets quantitatively: the greater the number of chimeric reads per target (labelled on the x-axis underneath the blue boxes) the deeper is target repression upon miR-1 transfection. The magnitude of repression increased among miR-eCLIP targets when more chimeric reads were identified (3, 10 or 25 chimeric reads per peak), indicating that the count of chimeric reads per target provides a quantitative metric that correlates with the strength of a particular mRNA-miRNA target interaction.

Finally, these results against TargetScan computationally predicted targets were compared. Although TargetScan-predicted targets did show significant repression upon miRNA over-expression, the magnitude was similar to only the low-confidence (>=3 read) chimeric eCLIP targets, with >=10 and >=25 read targets showed deeper repression upon miRNA over-expression (FIG. 41 and FIG. 42). Therefore, chimeric reads in miR-eCLIP libraries allow to vary a cutoff for a minimum number of chimeric reads per target adjusting sensitivity-specificity balance as well as provide a way to quantitively predict strength of miRNA targets.

1. A method of enriching microRNA (miRNA) targeted RNA molecules, wherein the method comprises:
contacting an RNA sample with target specific miRNA molecules in the presence of Argonaute 2 (Ago2) proteins to form a complex;
isolating the complex;
ligating the miRNA molecules to the RNA molecules within each complex to form chimeric RNA molecules;
enriching non-chimeric RNA molecules of interest and chimeric RNA molecules, or cDNA molecules thereof, with probes;
amplifying enriched non-chimeric RNA molecules and chimeric RNA molecules, or cDNA molecules thereof, by PCR;
sequencing the PCR products; and
identifying computationally non-chimeric RNA molecules of interest and/or chimeric RNA molecules.

2. (canceled)

3. The method of claim 1, further comprising lysing cells prior to isolating the complexes.

4. The method of claim 1, wherein contacting the RNA sample further comprises crosslinking the complex together by UV light or a chemical crosslink agent.

5. The method of claim 4, wherein the chemical crosslink agent is selected from formaldehyde, formalin, acetaldehyde, prionaldehyde, water-soluble carbomiidides, phenylglyoxal, and UDP-dialdehyde.

6. The method of claim 1, wherein the RNA sample comprises mRNA molecules or mRNA fragments.

7. The method of claim 1, wherein isolating the complex is by immunoprecipitation of the complex.

8. The method of claim 7, wherein the immunoprecipitation comprises contacting the complex with an Ago2 antibody.

9. The method of claim 8, wherein the contacting step is followed converting associated RNA into libraries that can be subjected to high-throughput sequencing to quantify association.

10. The method of claim 1, wherein the non-chimeric RNA molecules of interest are miRNA molecules.

11. The method of claim 10, wherein the probes are anti-sense nucleic acid probes in a length between 10 bp and 100 bp.

12. The method of claim 11, wherein the probes are 100% complementary to the miRNA molecules.

13. The method of claim 1, wherein the non-chimeric RNA molecules of interest map to specific genes or 3′-UTR of genes.

14. The method of claim 13, wherein the probes are anti-sense nucleic acid probes in a length between 10 bp and 5000 bp.

15. The method of claim 13, wherein the cDNA molecules are formed by reverse transcribing RNA molecules into the cDNA molecules before the enriching step.

16. The method of claim 11, wherein the probes are RNA, single stranded DNA (ssDNA), or synthetic nucleic acids, such as LNA.

17. The method of claim 1, further comprising digesting the Ago2 proteins prior to the enriching step.

18. The method of claim 1, wherein the enrichment step produces about 5% to about 30% chimeric reads out of all uniquely mapped reads.

19. The method of claim 1, wherein the enrichment step increases the proportion of chimeric reads in the library.

20. The method of claim 19, wherein the overall chimeric read population is increased by at least 20-fold.

21. (canceled)

22. (canceled)

23. (canceled)

24. (canceled)

25. The method of claim 1, wherein the Ago2 includes a gene selected from APP, ATG9A, BTG2, and ULK1.

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)


SEQUENCE LISTING (18 sequences)

SEQ ID NO: 1 - DNA, 30 bp, Artificial Sequence
  Artificially Synthesized Primer
  misc_feature (1)...(10): n = A, T, C or G
nnnnnnnnnnagatcggaagagcgtcgtgt

SEQ ID NO: 2 - RNA, 21 nt, Artificial Sequence
  Artificially Synthesized Primer
uucacaguggcuaaguuccgc

SEQ ID NO: 3 - RNA, 21 nt, Artificial Sequence
  Artificially Synthesized Primer
uucacaguggcuaaguucugc

SEQ ID NO: 4 - RNA, 23 nt, Artificial Sequence
  Artificially Synthesized Primer
agcuacauugucugcuggguuuc

SEQ ID NO: 5 - RNA, 21 nt, Artificial Sequence
  Artificially Synthesized Primer
agcuacaucuggcuacugggu

SEQ ID NO: 6 - RNA, 23 nt, Artificial Sequence
  Artificially Synthesized Primer
caaagugcuguucgugcagguag

SEQ ID NO: 7 - RNA, 23 nt, Artificial Sequence
  Artificially Synthesized Primer
caaagugcuuacagugcagguag

SEQ ID NO: 8 - RNA, 23 nt, Artificial Sequence
  Artificially Synthesized Primer
uaaagugcuuauagugcagguag

SEQ ID NO: 9 - RNA, 23 nt, Artificial Sequence
  Artificially Synthesized Primer
caaagugcuuacagucgagguag

SEQ ID NO: 10 - RNA, 23 nt, Artificial Sequence
  Artificially Synthesized Primer
caaagugcuuacagugcagguag

SEQ ID NO: 11 - RNA, 23 nt, Artificial Sequence
  Artificially Synthesized Primer
uaaggugcaucuagugcagauag

SEQ ID NO: 12 - RNA, 23 nt, Artificial Sequence
  Artificially Synthesized Primer
uaaagugcuuauagugcagguag

SEQ ID NO: 13 - DNA, 90 bp, Artificial Sequence
  Artificially Synthesized Primer
attcaacatcagtctgataagctaacaaccagctaagacactgccagcggaacttagccactgtgaagaaacccagcagacaatgtagct

SEQ ID NO: 14 - DNA, 27 bp, Artificial Sequence
  Artificially Synthesized Primer
ggggcggaacttagccactgtgaacac

SEQ ID NO: 15 - DNA, 27 bp, Artificial Sequence
  Artificially Synthesized Primer
ggtgcagaacttagccactgtgaacaa

SEQ ID NO: 16 - RNA, 12 nt, Artificial Sequence
  Artificially Synthesized Primer
uauaagcuaucg

SEQ ID NO: 17 - RNA, 10 nt, Artificial Sequence
  Artificially Synthesized Primer
uuacugugaa

SEQ ID NO: 18 - RNA, 10 nt, Artificial Sequence
  Artificially Synthesized Primer
uauguagcag